SEQUENCE LISTING (25 sequences)

SEQ ID NO: 1 - protein, 724 aa, adeno-associated virus serotype 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 2 - protein, 588 aa, adeno-associated virus serotype 5
TAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 3 - protein, 532 aa, adeno-associated virus serotype 5
MSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 4 - protein, 18 aa, Artificial Sequence
  Flexible linker
KESGSVSSEQLAQFRSLD

SEQ ID NO: 5 - protein, 14 aa, Artificial Sequence
  Flexible linker
EGKSSGSGSESKST

SEQ ID NO: 6 - protein, 8 aa, Artificial Sequence
  PON1_P1_ApoA1-epitope
GKQLNLKL

SEQ ID NO: 7 - protein, 7 aa, Artificial Sequence
  PON1 P2 ApoA1 epitope
TLSEKAK

SEQ ID NO: 8 - protein, 11 aa, Artificial Sequence
  LCAT s108 ApoA1 epitope
TYSVEYLDSSK

SEQ ID NO: 9 - protein, 6 aa, Artificial Sequence
  LCAT K249 ApoA1 epitope
LKEEQR

SEQ ID NO: 10 - protein, 11 aa, Artificial Sequence
  ABCA1 ApoA1 epitope 1
RYVWGGFAYLQ

SEQ ID NO: 11 - protein, 9 aa, Artificial Sequence
  epitope 1 r587
FEDMRYVWG

SEQ ID NO: 12 - protein, 9 aa, Artificial Sequence
  epitpe 1 q597
FAYLQDVVE

SEQ ID NO: 13 - protein, 30 aa, Artificial Sequence
  ABCA1 epitope 2
CPPGAGGLPPPQRKQNTADILQDLTGRNIS

SEQ ID NO: 14 - protein, 10 aa, Artificial Sequence
  C1477 surround epitope
MLPVCPPGAG

SEQ ID NO: 15 - protein, 9 aa, Artificial Sequence
  S1506 surround epitope
GRNISDYLV

SEQ ID NO: 16 - protein, 9 aa, Artificial Sequence
  apoB LDL-receptor binding epitope
RLTRKRGLK

SEQ ID NO: 17 - DNA, 19 bp, Artificial Sequence
  CMV promoter forward primer
aatgggcggtaggcgtgta

SEQ ID NO: 18 - DNA, 22 bp, Artificial Sequence
  CMV promoter reverse primer
aggcgatctgacggttcactaa

SEQ ID NO: 19 - DNA, 20 bp, Artificial Sequence
  CMV promoter probe
tgggaggtctatataagcag

SEQ ID NO: 20 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAEAVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLT

SEQ ID NO: 21 - DNA, 2414 bp, Artificial Sequence
  AAV vector construct comprising human FIX_Padua coding sequence operably linked to an LP1 promoter and SV40 polyA-site and flanked by wt AAV2 ITRs
tgggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctggaggggtggagtcgtgacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactgaattctagaccaccatgcagagggtgaacatgatcatggctgagagccctggcctgatcaccatctgcctgctgggctacctgctgtctgctgagtgcactgtgttcctggaccatgagaatgccaacaagatcctgaacaggcccaagagatacaactctggcaagctggaggagtttgtgcagggcaacctggagagggagtgcatggaggagaagtgcagctttgaggaggccagggaggtgtttgagaacactgagaggaccactgagttctggaagcagtatgtggatggggaccagtgtgagagcaacccctgcctgaatgggggcagctgcaaggatgacatcaacagctatgagtgctggtgcccctttggctttgagggcaagaactgtgagctggatgtgacctgcaacatcaagaatggcagatgtgagcagttctgcaagaactctgctgacaacaaggtggtgtgcagctgcactgagggctacaggctggctgagaaccagaagagctgtgagcctgctgtgccattcccatgtggcagagtgtctgtgagccagaccagcaagctgaccagggctgaggctgtgttccctgatgtggactatgtgaacagcactgaggctgaaaccatcctggacaacatcacccagagcacccagagcttcaatgacttcaccagggtggtggggggggaggatgccaagcctggccagttcccctggcaagtggtgctgaatggcaaggtggatgccttctgtgggggcagcattgtgaatgagaagtggattgtgactgctgcccactgtgtggagactggggtgaagatcactgtggtggctggggagcacaacattgaggagactgagcacactgagcagaagaggaatgtgatcaggatcatcccccaccacaactacaatgctgccatcaacaagtacaaccatgacattgccctgctggagctggatgagcccctggtgctgaacagctatgtgacccccatctgcattgctgacaaggagtacaccaacatcttcctgaagtttggctctggctatgtgtctggctggggcagggtgttccacaagggcaggtctgccctggtgctgcagtacctgagggtgcccctggtggacagggccacctgcctgctgagcaccaagttcaccatctacaacaacatgttctgtgctggcttccatgaggggggcagggacagctgccagggggactctgggggcccccatgtgactgaggtggagggcaccagcttcctgactggcatcatcagctggggggaggagtgtgccatgaagggcaagtatggcatctacaccaaagtctccagatatgtgaactggatcaaggagaagaccaagctgacctgactcgatgctttatttgtgaaatttgtgatgctattgctttatttgtaaccattataagctgcaataaacaagttaacaacaacaattgcattcattttatgtttcaggttcagggggaggtgtgggaggttttttaaactaggtcacgactccacccctccaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccca

SEQ ID NO: 22 - DNA, 23 bp, Artificial Sequence
  SEAP forward primer
gtacccagatgactacagccaag

SEQ ID NO: 23 - DNA, 23 bp, Artificial Sequence
  SEAP reverse primer
ggtggatctcgtatttcatgtct

SEQ ID NO: 24 - DNA, 19 bp, Artificial Sequence
  AAV forward primer
aatgggcggtaggcgtgta

SEQ ID NO: 25 - DNA, 22 bp, Artificial Sequence
  AAV reverse primer
aggcgatctgacggttcactaa

(19) United States
(12) Patent Application Publication
(10) Pub. No.: US 2024/0374755 A1
(43) Pub. Date: Nov. 14, 2024

(54) Adeno-associated virus vectors modified to bind High-Density Lipoprotein

(71) Applicant: uniQure biopharma B.V., Amsterdam (NL)

(72) Inventors: Sander Jan Hendrik Van Deventer, Amsterdam (NL); Sebastiaan Menno Bosma, Amsterdam (NL); - Anggakusuma, Amsterdam (NL)

(73) Assignee: uniQure biopharma B.V., Amsterdam (NL)

(21) Appl. No.: 18/564,623

(22) PCT Filed: Jun. 2, 2022
(86) PCT No.: PCT/EP2022/065106

§ 371 Date: Nov. 28, 2023

(30) Foreign Application Priority Data
Jun. 2, 2021 (EP) 21177449.2
Jun. 16, 2021 (EP) 21179856.6

(51) Int. Cl. A61K 48/00; C07K 14/005; C12N 15/86

(52) U.S. Cl. CPC A61K 48/0041; C07K 14/005; C12N 15/86; C12N 2750/14122; C12N 2750/14143; C12N 2750/14145; C12N 2750/14151

(57) ABSTRACT

The present invention relates to AAV vectors comprising a capsid protein that has been modified to bind to high-density lipoprotein (HDL) by insertion of an HDL-binding moiety into the capsid protein. The HDL-binding moiety can be an HDL-binding protein or domain thereof, such as a single domain antibody like a VHH domain, or the HDL-binding moiety can be an HDL-binding epitope, e.g. derived from an HDL- or ApoA1-binding protein. The HDL-binding moiety is preferably inserted into an exposed loop of the capsid protein. The HDL-binding moiety can be expressed on one, two or all three of the VP1, VP2 and VP3 capsid proteins. The AAV vectors of the invention that to bind to HDL show improved transduction efficiency of liver cells as well as improved spread transduction throughout the liver. The invention therefore further provides for the use of the AAV vectors of the invention in the treatment of conditions that can be treated by gene therapy of the liver.

### FIELD OF THE INVENTION

The present invention relates to the fields of biotechnology, medicine and gene therapy. The invention relates to an adeno-associated virus (AAV) vector comprising one or more high density lipoprotein (HDL)-binding moieties on one or more exposed loops of the capsid protein, associated compositions, pharmaceutical compositions and uses in treatments thereof.

### BACKGROUND OF THE INVENTION

Adeno-associated viruses (AAV) are considered non-pathogenic, low immunogenic viruses that have been successfully used as vectors for in vivo gene delivery in animal models and human studies. AAVs are small 20- to 25-nm, non-enveloped, 4.7-kb single-stranded DNA viruses with an icosahedral 60-mer capsid. In non-replicative recombinant AAVs designed for gene delivery to a target cell, the nucleotide sequence to be delivered replaces the viral capsid and replication genes.

The delivered nucleotide sequence may encode, for example, a fluorescent reporter, a replacement for a defective cellular gene, a protein or an RNA modulator of a specific cellular function, or a toxin/suicide factor. The vectors persist in an extrachromosomal state without integrating into the genome.

A limiting factor for the use of adeno-associated viruses (AAVs) as vectors in gene therapy is the broad tropism of AAV serotypes, i.e., the parallel infection of several cell types. Current techniques of improving target specificity of AAVs include the chemical conjugation of two Fab fragments (Bartlett et al. Nat Biotechnol, 1999, vol. 17, 181-6), the insertion of peptides into the capsid proteins, the fusion of DARPins to the N-terminus of the VP2 capsid protein (Münch et al. Mol Ther, 2013, vol. 21, 109-18), the use of AAV serotypes that are not recognized by neutralizing antibodies. However, AAVs produced by these strategies still only show insufficient transduction efficiency of AAV vectors due to inefficient uptake, uncoating and/or translocation of the viral genome to the nucleus of the host cell.

AAV serotypes have been identified that display a tissue-specific tropism, such as a hepatocyte-selective tropism, where the combination with intravenous administration allows effective biodistribution of AAV to the liver, resulting in transduction and therapeutic expression of transgene sequences by hepatocytes. However, despite earlier observations that intravenous administration of AAV can result in effective transgene expression by the specific target tissue, successful transduction showed an inversed correlation to the proximity of the site of administration.

There is, therefore, still a need for further improvement of effective tissue-specific transduction, in, for example, hepatocytes, and more specifically, hepatocytes that are more distantly located from the portal vein, which may thus increase the intrahepatic distribution of AAV therapy. Such improvement would thus be applicable to all tissues and consequently increase absolute levels of transgene expression at lower AAV doses.

### SUMMARY OF THE INVENTION

The present invention solves this problem by providing AAV vectors comprising a capsid protein that have been modified to display at least one HDL-binding moiety, which specifically binds to HDL, e.g. by having an affinity for apolipoprotein A1 (ApoA1). The HDL-binding moiety displayed by the modified capsid protein confers to the AAV vectors of the invention the ability to associate with HDL particles in the bloodstream, or it increases that ability, whereby the AAV vectors are co-transported with HDL particles to HDL-associated organs, such as the liver, kidneys, brain, pancreas, spleen, ovaries, testes and adrenals, thereby increasing AAV transduction efficacy in and spread in these tissues.

In a first aspect, the invention pertains to an adeno-associated virus (AAV) vector comprising a capsid protein that comprises at least one high-density lipoprotein (HDL)-binding moiety, wherein preferably the HDL-binding moiety is exogenous to the capsid protein. Preferably, the capsid protein that comprises an HDL-binding moiety is at least a VP1 capsid protein, or wherein the capsid protein that comprise an HDL-binding moiety are at least the VP2 and VP3 capsid proteins. Alternatively, the VP1, VP2 and VP3 capsid proteins each comprise an HDL-binding moiety.

In one embodiment, there is provided an AAV vector, wherein at least one of: a) an HDL-binding moiety is inserted into an exposed loop of the capsid protein, wherein preferably the exposed loop is at least one of the GH-L1 loop and the GH-L5 loop of the capsid protein; and, b) an HDL-binding moiety is fused onto the carboxy-terminus of the capsid protein.

In one embodiment, there is provided an AAV vector, wherein the HDL-binding moiety is an HDL-binding epitope or an HDL-binding protein or domain thereof, wherein preferably, the HDL-binding protein or domain thereof is an antibody, antibody fragment, scFv, Fv, Fab, (Fab′)2, single domain antibody (sdAb), vH or vL domain, camelid VHH domain, cartilaginous fish VNAR domain, DARPin, affibody, affilin, adnectin, affitin, repebody, fynomer, alphabody, avimer, atrimer, centyrin, pronectin or anticalin that specifically binds HDL.

In one embodiment, there is provided an AAV vector, wherein the HDL-binding moiety has at least one of the characteristics: a) the HDL-binding moiety, when inserted in the GH-L1 or the GH-L5 loop of the AAV5 VP1 amino acid sequence and expressed in at least the VP1 capsid protein of AAV5 capsids, after overnight incubation of the AAV5 capsids with an equimolar amount of human HDL, in an immunoprecipitation with an anti-human HDL antibody, causes the AAV5 capsids comprising the HDL-binding moiety to co-precipitate with human HDL for at least 10% of the AAV5 capsids or the HDL-binding moiety causes the ability of the AAV5 capsids comprising the HDL-binding moiety to co-precipitate with human HDL to increase by a factor 1.1, 1.2, 1.5, 2.0, 5.0, 10, 20, 50 or 100 as compared to corresponding wt AAV5 capsids; b) when inserted in the GH-L1 or the GH-L5 loop of the AAV5 VP1 amino acid sequence and expressed in at least the VP1 capsid protein of AAV5 capsids, after overnight incubation of the AAV5 capsids with an equimolar amount of human HDL, in an continuous iodixanol density gradient centrifugation has a lighter density than the density of corresponding wt AAV5 capsids for at least 10% of the AAV5 capsids; c) the HDL-binding moiety, when inserted in the GH-L1 or the GH-L5 loop of the AAV5 VP1 amino acid sequence and expressed in at least the VP1 capsid protein of an AAV5 vector, causes the AAV5 vector comprising the HDL-binding moiety to have an increased efficiency of transduction of at least a factor 1.1 as compared to a corresponding wt AAV5 vector when tested in vitro on a liver cell line or on primary human hepatocytes (PHH); d) the HDL-binding moiety, when inserted in the GH-L1 or the GH-L5 loop of the AAV5 VP1 amino acid sequence and expressed in at least the VP1 capsid protein of an AAV5 vector, causes the AAV5 vector comprising the HDL-binding moiety to have an increased efficiency of transduction of at least a factor 1.1 as compared to a corresponding wt AAV5 vector when tested in vitro on human iPSC-derived forebrain neurons; and, e) the HDL-binding moiety, when inserted in the GH-L1 or the GH-L5 loop of the AAV5 VP1 amino acid sequence and expressed in at least the VP1 capsid protein of an AAV5 vector, causes an increase in in vivo transduction of hepatocytes that are more distantly located from the portal vein by the AAV5 vector comprising the HDL-binding moiety as compared to a corresponding wt AAV5 vector, as determined in transgenic mice expressing human APOA1.

In one embodiment, there is provided an AAV vector, wherein the HDL-binding moiety is an Apolipoprotein A-I (ApoA1)-binding moiety, wherein preferably the ApoA1-binding moiety is an ApoA1-binding epitope derived from an ApoA1-binding protein selected from the group consisting of: PON1, LCAT, ABCA1 and apoB. Preferably, the ApoA-1-binding epitope comprises an amino acid sequence with no more than 1 or 2 amino acid differences per 6 amino acids from an amino acid sequence selected from the group consisting of the amino acid sequences of SEQ ID NO's: 6-16, and wherein preferably, the ApoA1-binding epitope is inserted in at least one of the VP1, VP2 and VP3 capsid proteins, between positions corresponding to positions T444 and G445 of SEQ ID NO: 1 and/or between positions corresponding to positions A579 and P580 of SEQ ID NO: 1, wherein more preferably the ApoA-1-binding epitope is expressed on all three of the VP1, VP2 and VP3 capsid proteins.

In one embodiment, there is provided an AAV vector, wherein at least one capsid protein is an AAV5 capsid protein.

In one embodiment, there is provided an AAV vector, comprising a nucleic acid molecule encapsidated by the capsid proteins, wherein the nucleic acid molecule comprises a transgene flanked by at least one AAV inverted terminal repeat (ITR).

In a further aspect, the invention relates to a composition comprising an AAV vector according to the invention, wherein preferably the composition is a pharmaceutical composition comprising the AAV vector and at least one pharmaceutically acceptable carrier.

In another aspect, the invention relates to an AAV vector according to the invention, or a composition according to the invention, for use as a medicament, wherein preferably the medicament is used in the treatment of a condition that can be treated by gene therapy of the liver or the central nervous system.

In yet another aspect, the invention relates to a nucleic acid molecule comprising a nucleotide sequence encoding a capsid protein that comprises at least one HDL-binding moiety as defined herein, wherein preferably nucleic acid molecule is an expression construct for expression of the capsid protein in a suitable host cell.

In again another aspect, the invention relates to a host cell comprising a nucleic acid molecule as defined above, and optionally comprising further nucleotide sequences for the expression of an AAV vector according to the invention.

In a final aspect, the invention relates to a method for producing an AAV vector according to the invention, the method comprising the steps of: a) culturing a host cell as defined herein under conditions such that the AAV vector is produced; and, b) optionally, one or more of recovery, purification and formulation of the AAV vector.

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the method.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

For the purposes of the present invention, the term “obtained” is considered to be a preferred embodiment of the term “obtainable”. If hereinafter e.g. an antibody is defined to be obtainable from a specific source, this is also to be understood to disclose an antibody which is obtained from this source.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g. about 10) preferably means that the value may be the given value (of 10) more or less 0.1% of the value.

As used herein, “an effective amount” is meant the amount of an agent required to ameliorate the symptoms of a disease relative to an untreated patient. The effective amount of active agent(s) used to practice the present invention for therapeutic treatment of, for example a cancer, varies depending upon the manner of administration, the age, body weight, and general health of the subject. Ultimately, the attending physician or veterinarian will decide the appropriate amount and dosage regimen. Such amount is referred to as an “effective” amount, which may be determined as genome copies per kilogram (GC/kg). Thus, in connection with the administration of a drug which, in the context of the current disclosure, is “effective against” a disease or condition indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatments, wherein such methods of treatment comprise the administration of the product for use.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using global alignment algorithms (e.g. Needleman Wunsch) which align the sequences optimally over the entire length, while sequences of substantially different lengths are preferably aligned using local alignment algorithms (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall length, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

As used herein, the term “selectively hybridizing”, “hybridizes selectively” and similar terms are intended to describe conditions for hybridization and washing under which nucleotide sequences at least 66%, at least 70%, at least 75%, at least 80%, more preferably at least 85%, even more preferably at least 90%, preferably at least 95%, more preferably at least 98% or more preferably at least 99% homologous to each other typically remain hybridized to each other. That is to say, such hybridizing sequences may share at least 45%, at least 50%, at least 55%, at least 60%, at least 65, at least 70%, at least 75%, at least 80%, more preferably at least 85%, even more preferably at least 90%, more preferably at least 95%, more preferably at least 98% or more preferably at least 99% sequence identity.

A preferred, non-limiting example of such hybridization conditions is hybridization in 6× sodium chloride/sodium citrate (SSC) at about 45° C., followed by one or more washes in 1×SSC, 0.1% SDS at about 50° C., preferably at about 55° C., preferably at about 60° C. and even more preferably at about 65° C.

Highly stringent conditions include, for example, hybridization at about 68° C. in 5×SSC/5×Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), Sambrook and Russell (2001) “Molecular Cloning: A Laboratory Manual (3^{rd} edition), Cold Spring Harbor Laboratory, Cold Spring Harbor Laboratory Press, New York 1995, Current Protocols in Molecular Biology, (John Wiley & Sons, N.Y.).

Of course, a polynucleotide which hybridizes only to a poly A sequence (such as the 3′ terminal poly(A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly(A) stretch or the complement thereof (e.g., practically any double-stranded cDNA clone).

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. A “vector” is a nucleic acid construct (typically DNA or RNA) that serves to transfer an exogenous nucleic acid sequence (i.e. DNA or RNA) into a host cell. A vector is preferably maintained in the host by at least one of autonomous replication and integration into the host cell's genome. The terms “expression vector” or “expression construct” refer to nucleotide sequences that are capable of affecting expression of a gene in host cells or host organisms compatible with such sequences. These expression vectors typically include at least one “expression cassette” that is the functional unit capable of affecting expression of a sequence encoding a product to be expressed and wherein the coding sequence is operably linked to the appropriate expression control sequences, which at least comprises a suitable transcription regulatory sequence and optionally, 3′ transcription termination signals. Additional factors necessary or helpful in affecting expression may also be present, such as expression enhancer elements. The expression vector will be introduced into a suitable host cell and be able to affect expression of the coding sequence in an in vitro cell culture of the host cell. A preferred expression vector will be suitable for expression of viral proteins and/or nucleic acids, particularly recombinant AAV proteins and/or nucleic acids.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer or biological entity.

The term “reporter” may be used interchangeably with marker, although it is mainly used to refer to visible markers, such as green fluorescent protein (GFP) or luciferase.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which they are introduced but have been obtained from another cell or are synthetically or recombinantly produced. Generally, though not necessarily, such nucleic acids encode proteins, i.e. exogenous proteins, that are not normally produced by the cell in which the DNA is transcribed or expressed. Similarly, exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other.

As used herein, the term “non-naturally occurring” when used in reference to an organism means that the organism has at least one genetic alternation that is not normally found in a naturally occurring strain of the referenced species, including wild-type strains of the referenced species. Genetic alterations include, for example, modifications introducing expressible nucleic acids encoding proteins or enzymes, other nucleic acid additions, nucleic acid deletions, nucleic acid substitutions, or other functional disruption of the organism's genetic material. Such modifications include, for example, coding regions and functional fragments thereof for heterologous or homologous polypeptides for the referenced species. Additional modifications include, for example, non-coding regulatory regions in which the modifications alter expression of a gene or operon. Genetic modifications to nucleic acid molecules encoding enzymes, or functional fragments thereof, can confer a biochemical reaction capability or a metabolic pathway capability to the non-naturally occurring organism that is altered from its naturally occurring state.

As used herein, the term “operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signal for introns, and stop codons.

The term “expression control sequence” is intended to include, at a minimum, a sequence whose presence is designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA tail, i.e., a string of adenine residues at the 3′-end of a mRNA, sequences referred to as polyA sequences. It also can be designed to enhance mRNA stability. Expression control sequences which affect the transcription and translation stability, e.g., promoters, as well as sequences which affect the translation, e.g., Kozak sequences, are known in insect cells. Expression control sequences can be of such nature as to modulate the nucleotide sequence to which it is operably linked such that lower expression levels or higher expression levels are achieved.

### DETAILED DESCRIPTION OF THE INVENTION

The present inventors have set out to develop AAV vectors comprising capsid proteins that have been modified to contain at least one high-density lipoprotein (HDL)-binding moiety which binding moiety confers to the AAV vector the ability to associate with HDL particles, or increases the ability of the AAV vector to associate with HDL particles, which results in an AAV vector with enhanced capacity to transduce specific target cells and in an increased spread over specific target organs, such as the liver, brain, kidneys, pancreas, spleen, ovaries, testes and adrenals.

In a first aspect of the invention there is provided an adeno-associated virus (AAV) vector comprising a capsid protein that comprises at least one HDL-binding moiety. In one embodiment, the at least one HDL-binding moiety comprises or consists of an amino acid sequence that is exogenous to the amino acid sequence of the AAV capsid protein. As such, the at least one HDL-binding moiety is an exogenous HDL-binding moiety and a capsid protein comprising such exogenous HDL-binding moiety is a non-naturally occurring capsid protein. An amino acid sequence that is exogenous to the amino acid sequence of an AAV capsid protein is herein understood as an amino acid sequence that differs from an amino acid sequence of a wild type or parent AAV capsid protein by the insertion, substitution and/or deletion of one or more amino acids.

### Adeno-Associated Virus (AAV) Vectors

The present invention relates to recombinant parvoviruses, in particular dependoviruses such as infectious human or simian adeno-associated virus (AAV), and the components thereof (e.g. a parvovirus genome) for use as vectors for introduction and/or expression of nucleic acids in mammalian cells, preferably human cells. In particular, the invention relates to such AAV vectors that are modified in one or more of their capsid proteins to comprise at least one HDL-binding moiety, wherein preferably the at least one HDL-binding is exogenous to the one or more capsid proteins.

An “AAV vector” is defined as a recombinantly produced AAV or AAV particle that comprises a polynucleotide to be delivered into a host cell, either in vivo, ex vivo or in vitro. Herein, an AAV vector construct refers to the polynucleotide comprising the viral genome or part thereof, usually at least one ITR, and a transgene. Viruses of the Parvoviridae family are small DNA viruses. The family Parvoviridae may be divided between two subfamilies: the Parvovirinae, which infect vertebrates, and the Densovirinae, which infect invertebrates, including insects. Members of the subfamily Parvovirinae are herein referred to as the parvoviruses and include the genus Dependovirus. As may be deduced from the name of their genus, members of the Dependovirus are unique in that they usually require coinfection with a helper virus such as adenovirus or herpes virus for productive infection in cell culture. The genus Dependovirus includes AAV, which normally infects humans (e.g., serotypes 1, 2, 3A, 3B, 4, 5, 6, 7, 8, 9, 10, 11, 12 and 13) or primates (e.g., serotypes 1 and 4), and related viruses that infect other warm-blooded animals (e.g., bovine, canine, equine, and ovine adeno-associated viruses). Further information on parvoviruses and other members of the Parvoviridae is described in Kenneth I. Berns, “Parvoviridae: The Viruses and Their Replication,” Chapter 69 in Fields Virology (3d Ed. 1996). For convenience, the present invention is further exemplified and described herein by reference to AAV. It is however understood that the invention is not limited to AAV but may equally be applied to other parvoviruses.

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural viral particle (VP) proteins. The VP proteins (VP1, -2 and -3) form the capsid. The terminal 145 nt ITRs are self-complementary and are organized so that an energetically stable intramolecular duplex forming a T-shaped hairpin may be formed. These hairpin structures function as an origin for viral DNA replication, serving as primers for the cellular DNA polymerase complex. Following wild type (wt) AAV infection in mammalian cells the Rep genes (i.e. Rep78 and Rep52) are expressed from the P5 promoter and the P19 promoter, respectively, and both Rep proteins have a function in the replication and packaging of the viral genome. A splicing event in the Rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production. The three capsid proteins, VP1, VP2 and VP3 are expressed from a single VP reading frame from the p40 promoter. In mammalian cells wt AAV infection relies for the capsid proteins production on a combination of alternate usage of two splice acceptor sites and the suboptimal utilization of an ACG initiation codon for VP2. Rep 78 and Rep 68 have site-specific, single-strand endonuclease, DNA helicase, and ATP activities, respectively, which are responsible for DNA replication. Rep52 and Rep40 are responsible for packaging of DNA flanked by ITRs into capsids. The single Cap reading frame encodes for three capsid proteins: VP1, VP2 and VP3, which together assemble into the AAV icosahedral capsid. Additionally, the VP1 mRNA encodes for the assembly-activating protein (AAP) in a different reading frame.

A “recombinant parvoviral or AAV vector” (or “rAAV vector”) or a “parvoviral or AAV vector” herein refers to a parvoviral or AAV virion (i.e. a capsid), comprising (or “packaging”) one or more nucleotide sequences of interest, genes of interest or “transgenes” that is/are flanked by at least one parvoviral or AAV inverted terminal repeat sequence (ITR). Preferably, the transgene(s) is/are flanked by ITRs, one on each side of the transgene(s). Such (r)AAV vectors can be replicated and packaged into infectious viral particles when present in a suitable host cell that is expressing AAV rep and cap gene products (i.e. AAV Rep and Cap proteins). When the transgene(s) of interest that is/are flanked by at least one ITR is incorporated into a larger nucleic acid construct (e.g. in a chromosome or in another vector such as a plasmid or baculovirus used for cloning or transfection), this is typically referred to as a “pro-vector” which can be “rescued” by replication and encapsidation in the presence of AAV packaging functions and necessary helper functions.

### Capsid Proteins

The AAV icosahedral capsid with a diameter of about 260 Å is assembled from 60 capsid VP monomers consisting of three VPs: VP1, VP2, and VP3. VP3 is the major capsid protein, accounting for approximately 50 of the 60 capsid monomers, while there are approximately 5 copies each of VP1 and VP2 (and thus a ratio of 1:1:10 for VP1: VP2: VP3). An AAV vector of the invention comprises at least one capsid protein comprises at least one HDL-binding moiety. Suitable HDL-binding moieties for incorporation into AAV capsid protein in accordance with the invention are further specified in more detail herein below. An AAV vector of the invention can thus comprise an HDL-binding moiety in one, two or all three of the VP1, VP2 and VP3 capsid proteins.

In one embodiment, an AAV vector of the invention comprises an HDL-binding moiety in at least a VP1 capsid protein. In one embodiment, the AAV vector comprises an HDL-binding moiety in only a VP1 capsid protein, i.e. not in VP2 and VP3 capsid proteins. In one embodiment, the HDL-binding moiety is exogenous to the VP1 capsid protein. Such VP1 capsid protein is thus a non-naturally occurring capsid protein.

In one embodiment, an AAV vector of the invention comprises an HDL-binding moiety in at least the VP2 and VP3 capsid proteins. In one embodiment, the AAV vector comprises an HDL-binding moiety in only the VP2 and VP3 capsid proteins, i.e. not in a VP1 capsid protein. In one embodiment, the HDL-binding moiety is exogenous to the VP2 and VP3 capsid proteins. Such VP2 and VP3 capsid proteins are thus a non-naturally occurring capsid proteins.

In one embodiment, an AAV vector of the invention comprises an HDL-binding moiety in at least a VP2 capsid protein. In one embodiment, the AAV vector comprises an HDL-binding moiety in only a VP2 capsid protein, i.e. not in the VP1 and VP3 capsid proteins. In one embodiment, the HDL-binding moiety is exogenous to the VP2 capsid protein. Such VP2 capsid protein is thus a non-naturally occurring capsid protein.

In one embodiment, an AAV vector of the invention comprises an HDL-binding moiety in at least a VP3 capsid protein. In one embodiment, the AAV vector comprises an HDL-binding moiety in only a VP3 capsid protein, i.e. not in the VP1 and VP2 capsid proteins. In one embodiment, the HDL-binding moiety is exogenous to the VP3 capsid protein. Such VP3 capsid protein is thus a non-naturally occurring capsid protein.

In one embodiment, an AAV vector of the invention comprises at least one HDL-binding moiety in each of the VP1, VP2 and VP3 capsid proteins. In one embodiment, the HDL-binding moiety is exogenous to the VP1, VP2 and VP3 capsid proteins. Such VP1, VP2 and VP3 capsid proteins are thus a non-naturally occurring capsid proteins.

AAV is able to infect a number of mammalian cells, see, e.g., Tratschin et al. (1985, Mol. Cell Biol. 5:3251-3260) and Grimm et al. (1999, Hum. Gene Ther. 10:2445-2450). However, the cellular tropicity of AAV differs among serotypes. See, e.g., Davidson et al. (2000, Proc. Natl. Acad. Sci. USA, 97:3428-3432), who discuss differences among AAV2, AAV4, and AAV5 with respect to mammalian CNS cell tropism and transduction efficiency. In addition to cellular tropicity, AAV serotypes also differ in the seroprevalences of neutralizing antibodies and also in the cross-reactivity of such neutralizing antibodies towards different AAV serotypes (See e.g. Boutin et al. Hum Gene Ther. 2010 June; 21(6):704-12). Based on these differences a skilled person will know how to select the serotype of the capsid proteins of an AAV vector of the invention.

Thus, sequences coding for the VP1, and/or VP2 and VP3 capsid proteins for use in the context of the present invention can be taken from any of the known 42 serotypes, more preferably from an AAV which normally infects humans, such as AAV1, AAV2, AAV3, AAV4, AAV5, AAV6, AAV7, AAV8. AAV9, AAV10, AAV11, AAV12 or AAV13 or newly developed capsid proteins obtained by e.g. capsid shuffling techniques and AAV capsid libraries, or from newly and synthetically designed, developed or evolved capsid, such as the Anc-80 capsid.

Without wishing to be bound by theory, incorporating HDL-binding proteins on the surface of AAV vectors of any serotype may be used to de-target the AAVs natural tropism, and re-target them towards HDL-associated organs, such as the liver and brain. Binding of HDL particles to HDL-binding proteins expressed on the surface of the AAV vectors of the invention would block surface binding sites and prevent access of the regular cell surface receptors on the cell to the AAV vector, facilitating the re-targeting of the AVV-HDL vectors towards HDL-associated organs.

In one embodiment, an AAV vector of the invention comprises capsid proteins that are of a serotype selected from the group consisting of AAV1, AAV2, AAV3A, AAV3B, AAV4, AAV5, AAV6, AAV7, AAV8, AAV9, AAV10, AAV11, AAV12, AAV13 and AAVrh10. In a preferred embodiment, the AAV vector comprises capsid proteins that are of a serotype selected from the group consisting of AAV1, AAV2, AAV5, AAV8 and AAV9, of which AAV5 is most preferred. Amino acid sequences of the AAV5 VP1, VP2 and VP3 capsid protein are presented in SEQ ID NO's: 1-3, respectively.

In one embodiment, an AAV vector of the invention comprises capsid proteins having the amino acid sequences of the wild type AAV serotypes as indicated above (except for the one or more HDL-binding moieties). Alternatively, the capsid amino acid sequences and the nucleotide sequences encoding them can be man-made, for example, the sequence may be a hybrid form or may be codon optimized, such as for example by codon usage of AcmNPv or Spodoptera frugiperda. It is understood that the exact molecular weights of the capsid proteins, as well as the exact positions of the translation initiation codons may differ between different parvoviruses. However, the skilled person will know how to identify the corresponding position in nucleotide sequence from other parvoviruses than AAV5. Alternatively, the sequence encoding AAV capsid proteins is a man-made sequence, for example as a result of directed evolution experiments. This can include generation of capsid libraries via DNA shuffling, error prone PCR, bioinformatics rational design, site saturated mutagenesis. Resulting capsids are based on the existing serotypes but contain various amino acid or nucleotide changes that improve the features of such capsids. The resulting capsids can be a combination of various parts of existing serotypes, “shuffled capsids” or contain completely novel changes, i.e. additions, deletions or substitutions of one or more amino acids or nucleotides, organized in groups or spread over the whole length of gene or protein. See for example Schaffer and Maheshri; Proceedings of the 26th Annual International Conference of the IEEE EMBS San Francisco, CA, USA; Sep. 1-5, 2004, pages 3520-3523; Asuri et al., 2012, Molecular Therapy 20(2):329-3389; Lisowski et al., 2014, Nature 506(7488):382-386, herein incorporated by reference.

The at least one HDL-binding moiety can in principle be incorporated in any position in the amino acid sequence of a capsid protein as comprised in an AAV vector of the invention, and/or the at least one moiety can be attached to either end of the amino acid sequence of a capsid protein. However, preferably an HDL-binding moiety is inserted in or attached to the amino acid sequence of a capsid protein such that the moiety is exposed on the surface of the capsid so as to be available for binding to HDL. Therefore, in one embodiment, in the AAV vector according to the invention, at least one of: a) an HDL-binding moiety is inserted into an exposed loop of a capsid protein; and, b) an HDL-binding moiety is fused onto the carboxy-terminus of a capsid protein. Thus, in one embodiment, there is provided the AAV vector according to the invention, wherein at least one HDL-binding moiety is inserted into an exposed loop of a capsid protein. In an alternative embodiment, there is provided the AAV vector according to the invention, wherein at least one HDL-binding moiety is fused onto the carboxy-terminus of a capsid protein, which is also located near the surface of the capsid. Consequently, in one embodiment, there is provided, the AAV vector according to the invention, wherein at least one HDL-binding moiety is inserted into an exposed loop of a capsid protein and wherein is at least one HDL-binding moiety fused onto the carboxy-terminus of a capsid protein. In a preferred embodiment of an AAV vector according to the invention, at least one HDL-binding moiety is inserted into an exposed loop of a capsid protein of the vector.

X-ray crystallographic studies have shown that the icosahedral 3-fold axis of a parvoviral capsid, such as that of AAV5, is surrounded by protrusions to which two finger-like VP loops contribute (Govindasamy et al., J Virol. 2013, 87(20):11187-99. doi: 10.1128/JVI.00867-13; Zhang et al., Nat Commun. 2019, 10(1):3760. doi: 10.1038/s41467-019-11668-x). These two finger-like VP loops, termed GH-L1 and GH-L5, form the two outmost protrusions of the capsid exterior. Residues located within GH-L1 and GH-L5 (variable regions (VR) IV and VIII respectively) were shown to be involved in receptor attachment and transduction. It has however been demonstrated that the GH-L1 and GH-L5 loops can accommodate insertions without compromising infectivity of the resulting virions. In one embodiment therefore, there is provided an AAV vector comprising an HDL-binding moiety inserted into at least one of the GH-L1 loop and the GH-L5 loop of a parvoviral capsid protein, of which the GH-L1 loop is preferred. Preferably, the HDL-binding moiety is inserted into at least one of the GH-L1 loop and the GH-L5 loop of at least the parvoviral VP1 capsid protein, whereby the GH-L1 loop is preferred. More preferably, the HDL-binding moiety is inserted into at least one of the GH-L1 loop and the GH-L5 loop of only the parvoviral VP1 capsid protein, whereby the GH-L1 loop is preferred.

In one embodiment, there is provided an AAV vector comprising an HDL-binding moiety inserted in the capsid protein in the GH-L1 loop in the variable region (VR) IV, which, with reference to the amino acid sequence of the AAV5 VP1 capsid protein (SEQ ID NO: 1), comprises amino acid positions F438-F449 (corresponding to amino acid positions L445-F462 in VP1 of AAV2). In a preferred embodiment, the HDL-binding moiety is inserted into the VR IV of at least or only the VP1 capsid protein. In one embodiment, there is provided an AAV vector comprising an HDL-binding moiety inserted in the capsid protein in the GH-L1 loop between T444 and G445, between G445 and G446, or between G446 and V447, of which G445 and G446 are preferred. In a preferred embodiment, the HDL-binding moiety is inserted in the GH-L1 loop between T444 and G445, between G445 and G446, or between G446 and V447 (of which G445 and G446 are preferred) of at least or only the VP1 capsid protein, preferably of all three capsid proteins.

In one embodiment, there is provided an AAV vector comprising an HDL-binding moiety is inserted in the GH-L5 loop in the VR VIII, which, with reference to the amino acid sequence of the AAV5 VP1 capsid protein (SEQ ID NO: 1), comprises amino acid positions Q574-P580 (corresponding to amino acid positions Q584-A590 in VP1 of AAV2). In a preferred embodiment, the HDL-binding moiety is inserted into the VR VIII of at least or only the VP1 capsid protein. In one embodiment, there is provided an AAV vector comprising an HDL-binding moiety is inserted in the GH-L5 loop between Q574 and S575, between S575 and S576, between S576 and T577, between T577 and T578, between T578 and A579, or between A579 and P580. In a preferred embodiment, the HDL-binding moiety is inserted in the GH-L5 loop between Q574 and S575, between S575 and S576, between S576 and T577, between T577 and T578, between T578 and A579, or between A579 and P580 of at least or only the VP1 capsid protein, preferably of all three capsid proteins.

Corresponding positions for the insertion of an HDL-binding moiety in the GH-L1 or GH-L5 loops, respectively VR IV or VR VIII, of capsid proteins of other AAV serotypes, or of other parvoviruses, as indicated throughout this document, can be identified by alignment of the VP1 amino acid sequences with that of the AAV5 VP1, e.g. using an alignment algorithm and settings as herein described above.

It is further understood that the insertion of HDL-binding moiety as described herein above can be an insertion in the strict sense, i.e. without the removal of any native amino acid residues from the amino acid sequence of the capsid protein. However, also include in the invention is the insertion of an HDL-binding moiety with the concomitant replacement of 1, 2, 3, 4, 5, 6, 7, 8, 9, 10, 11, 12, 13, 14, 15, 16, 17, 18, 19, 20, 22, 25, or 30 native amino acid residues from the amino acid sequence of the capsid protein, whereby the native amino acid residues are preferably replaced with the HDL-binding moiety.

In one embodiment, the at least one HDL-binding moiety is inserted into at least one of the GH-L1 loop and the GH-L5 loop of the VP1, VP2 and VP3 capsid proteins. Thereby, each AAV particle exposes at least 60 HDL-binding moieties at its surface.

However, in this regard it is noted that insertion of an HDL-binding moiety into all three parvoviral capsid proteins is preferred only for HDL-binding moieties of smaller size, e.g. <40 or 30 amino acids, such as the HDL-binding epitopes described herein below. Insertion of larger HDL-binding moieties (e.g. >50 or 100 amino acids, such as HDL-binding proteins or domains thereof as described herein below) into all three parvoviral capsid proteins can interfere with capsid assembly and/or other viral functions, such as e.g. infectivity, due to steric hindrance by the inserted larger HDL-binding moieties. It is therefore preferred that an HDL-binding moiety of more than 40, 50, 60, 80 or 100 amino acids in length are is inserted only in a VP1 or VP2 capsid protein, of which VP1 is preferred. During assembly, VP1, VP2, and VP3 are incorporated at a ratio of resp. 1:1:10 into parvoviral capsids. Insertion of the exogenous amino acid sequence into only a VP1 or VP2 capsid protein therefore does not interfere with capsid assembly or infectivity.

In one embodiment, an HDL-binding moiety that is inserted in a parvoviral capsid protein comprises a linker sequence on at least one of the N-terminal or C-terminal end of the amino acid sequence as it is inserted. In one embodiment, the linker sequence is a flexible linker sequence. Suitable flexible linker-amino acid sequences are known in the art (e.g. from Chen et al., 2013, Adv Drug Deliv Rev. 65(10): 1357-1369). Flexible linkers are usually applied when the joined domains require a certain degree of movement or interaction. They are generally composed of small, non-polar (e.g. Gly) or polar (e.g. Ser or Thr) amino acids. The small size of these amino acids provides flexibility, and allows for mobility of the connecting functional domains. The incorporation of Ser or Thr can maintain the stability of the linker in aqueous solutions by forming hydrogen bonds with the water molecules, and therefore reduces the unfavorable interaction between the linker and the protein moieties. Preferred flexible linkers have sequences consisting primarily of stretches of Gly and Ser residues (“GS” linker). An example of preferred (and widely used) flexible linker has the sequence of (Gly-Gly-Gly-Gly-Ser) n. By adjusting the copy number “n”, the length of this GS linker can be optimized to achieve appropriate separation of the functional domains, or to maintain necessary inter-domain interactions. In one embodiment, n is 1, 2, 3, 4, or 5. Besides the GS linkers, many other flexible linkers have been designed for recombinant fusion proteins. These flexible linkers are also rich in small or polar amino acids such as Gly and Ser, but can contain additional amino acids such as Thr and Ala to maintain flexibility, as well as polar amino acids such as Lys and Glu to improve solubility, such as e.g. the flexible linkers having the amino acid sequences of SEQ ID NO's: 4 and 5, that have been applied for the construction of a bioactive scFv's.

### HDL-Binding Moieties

HDL is found throughout the body and may enter organs, such as the liver, kidney, brain, pancreas, spleen, ovaries, testes and adrenals, from blood or cerebral spinal fluid via the HDL-receptor. While liver is the main target organ to which HDL is transported, and brain the major off-target organ, with HDL maturing mostly on the brain side of the blood brain barrier (BBB), the kidney, pancreas, spleen, ovaries, testes and adrenals play a key role in the production of HDL particles, which are subsequently transported away from them. As an example, HDL is one of the main lipid transporters that moves fatty acids, cholesterol and phospholipids from peripheral cells to the liver. As such, providing an AAV vector with affinity for HDL in accordance with the present invention allows the AAV vector to piggy-bag onto HDL as a natural carrier of the vector towards the liver, thereby effecting a better spread of the vector in the liver. In accordance with the present invention therefore, there is provided an AAV vector comprising a capsid protein that comprises at least one HDL-binding moiety.

The term “HDL-binding moiety”, as used herein, refers to any amino acid sequence capable of specifically binding to HDL, i.e. having HDL binding affinity. The term “HDL-binding moiety” thus includes, without limitation, HDL-binding epitopes, including both naturally-occurring and synthetic HDL-binding epitopes (as further defined below), as well as HDL-binding proteins or HDL-binding domains thereof. Such as HDL-binding proteins or domains can be e.g. an antibody or antibody fragment, an scFv, a Fv, a Fab, a (Fab′)2, a single domain antibody (sdAb), a vH or vL domain, a camelid VHH domain, a cartilaginous fish VNAR domain, a non-immunoglobulin scaffold such as a DARPIN, an affibody, an affilin, an adnectin, an affitin, a repebody, a fynomer, an alphabody, an avimer, an atrimer, a centyrin, a pronectin, or an anticalin.

By “specific binding” is meant that the binding is selective for HDL or a part thereof (e.g. ApoA1) and can be discriminated from unwanted or non-specific interactions. The ability of an HDL-binding moiety to bind to HDL or a part thereof can be measured either through an enzyme-linked immunosorbent assay (ELISA) or other techniques familiar to one of skill in the art, e.g. surface plasmon resonance (SPR) technique (analyzed e.g. on a BIAcore instrument) (Liljeblad et al., Glyco J 17, 323-329 (2000)), and traditional binding assays (Heeley, Endocr Res 28, 217-229 (2002)). In one embodiment, the extent of binding of an HDL-binding moiety to an unrelated protein is less than about 10% of the binding of the antigen binding moiety to the antigen as measured, e.g., by SPR.

In one embodiments, an HDL-binding moiety has a dissociation constant (K_{D}) of at least 10 μM, 1 μM, at least 100 nM, at least 10 nM, at least 1 nM, at least 0.1 nM, at least 0.01 nM or at least 0.001 nM.

In one embodiment, the HDL-binding moiety has one or more of the further characteristics.

Without wishing to be bound by theory, a wt AAV capsid may already have some tendency to associate with the lipid fraction of blood or serum. Therefore, in one embodiment, the HDL-binding moiety, when expressed at the surface of an AAV vector by insertion of the HDL-binding moiety into an exposed loop of at least one an AAV capsid protein, confers to the AAV vector the ability to associate with HDL particles, or increases the ability of the AAV vector to associate with HDL particles. The ability of an HDL-binding moiety to confer to an AAV vector the ability to associate with HDL particles, or to increase that ability, can be determined in one or more of the following assays.

In one embodiment, the HDL-binding moiety, when inserted in the GH-L1 or the GH-L5 loop of the AAV5 VP1 amino acid sequence and expressed in at least the VP1 capsid protein of AAV5 capsids, after overnight incubation of the AAV5 capsids with an equimolar amount of human HDL, in an immunoprecipitation with an anti-human HDL antibody, causes the AAV5 capsids comprising the HDL-binding moiety to co-precipitate with human HDL for at least 10, 20, 30, 40, 50, 50, 60, 70, 80, 90, 95, or 98% of the AAV5 capsids or the HDL-binding moiety causes the ability of the AAV5 capsids comprising the HDL-binding moiety to co-precipitate with human HDL to increase by a factor 1.1, 1.2, 1.5, 2.0, 5.0, 10, 20, 50 or 100 as compared to corresponding wt AAV5 capsids.

In one embodiment, the HDL-binding moiety, when inserted in the GH-L1 or the GH-L5 loop of the AAV5 VP1 amino acid sequence, and expressed in at least the VP1 capsid protein of AAV5 capsids, after overnight incubation of the AAV5 capsids with an equimolar amount of human HDL, in an continuous iodixanol density gradient centrifugation causes the AAV5 capsids comprising the HDL-binding moiety to have a lighter density than the density of corresponding wt AAV5 capsids (e.g. 1.21 g/cm³) for at least 10, 20, 30, 40, 50, 50, 60, 70, 80, 90, 95, or 98% of the AAV5 capsids.

In one embodiment, the HDL-binding moiety, when inserted in the GH-L1 or the GH-L5 loop of the AAV5 VP1 amino acid sequence and expressed in at least the VP1 capsid protein of an AAV5 vector, causes the AAV5 vector comprising the HDL-binding moiety to have an increased efficiency of transduction of at least a factor 1.1, 1.2, 1.5, 2.0, 5.0, 10, 15, 20, 50 or 100 as compared to a corresponding wt AAV5 vector when tested in vitro on a liver, kidney, brain, pancreas, spleen, ovary, testis or adrenals cell line or on primary human equivalent cell.

In one embodiment, the HDL-binding moiety, when inserted in the GH-L1 or the GH-L5 loop of the AAV5 VP1 amino acid sequence and expressed in at least the VP1 capsid protein of an AAV5 vector, causes the AAV5 vector comprising the HDL-binding moiety to have an increased efficiency of transduction of at least a factor 1.1, 1.2, 1.5, 2.0, 5.0, 10, 15 or 20 as compared to a corresponding wt AAV5 vector when tested in vitro on a liver cell line or on primary human hepatocytes (PHH). Preferably, the liver cell line is the Huh7 cell line or the HepG2 cell line. Transduction efficiency in in vitro liver cell culture is preferably determined after 48 hours.

In one embodiment, the HDL-binding moiety, when inserted in the GH-L1 or the GH-L5 loop of the AAV5 VP1 amino acid sequence, and expressed in at least the VP1 capsid protein of an AAV5 vector, causes the AAV5 vector comprising the HDL-binding moiety to have an increased efficiency of transduction of at least a factor 1.1, 1.2, 1.5, 2.0, 5.0, 10, 15 or 20 as compared to a corresponding wt AAV5 vector when tested in vitro on human iPSC-derived forebrain neurons. Transduction efficiency in in vitro human iPSC-derived forebrain neurons is preferably determined after 72 hours.

The difference in efficiency of transduction between an AAV vector of the invention and a corresponding wt AAV5 vector in in vitro cell culture can be determined by methods well known to the skilled person, e.g. by determining AAV genome copies/ml and/or determining the relative expression level of the transgene in the AAV vector. Preferably the efficiency of transduction in in vitro cell culture is determined at an multiplicity of infection of one or more of 104, 105 and 106.

In one embodiment, the HDL-binding moiety, when inserted in the GH-L1 or the GH-L5 loop of the AAV5 VP1 amino acid sequence, and expressed in at least the VP1 capsid protein of an AAV5 vector, causes an increase in in vivo transduction of cells that are more distantly located from the site of administration by the AAV5 vector comprising the HDL-binding moiety as compared to a corresponding wt AAV5 vector.

In one embodiment, the HDL-binding moiety, when inserted in the GH-L1 or the GH-L5 loop of the AAV5 VP1 amino acid sequence, and expressed in at least the VP1 capsid protein of an AAV5 vector, causes an increase in in vivo transduction of hepatocytes that are more distantly located from the portal vein by the AAV5 vector comprising the HDL-binding moiety as compared to a corresponding wt AAV5 vector, as determined in transgenic mice expressing human APOA1.

While HDL is a complex particle that consists of multiple proteins and phospholipids, its main protein component is ApoA1. Therefore, in one embodiment, there is provided an AAV vector according to the invention, wherein the HDL-binding moiety is an Apolipoprotein A-I (ApoA1)-binding moiety.

ApoA1 (a biphasic protein) is a dimer that wraps around the phospholipids, presenting its apolar side to the lipids and its polar side to outside. ApoA1 also is the main docking molecule for proteins interacting with HDL e.g. during phospholipid loading. Proteins known to interact with HDL and/or ApoA1 include e.g. PON1, LCAT, ABCA1 and ApoB. Such proteins thus comprises regions within their amino acid sequences with affinity for HDL, or more specifically with affinity for ApoA1. Such a region within an amino acid that confers affinity for HDL and/or ApoA1 is herein thus defined as an “HDL-binding epitope”. An HDL-binding epitope will usually consists of relatively short, e.g. 5 to 35, or 6 to 30 amino acid sequences that can confer affinity for HDL and/or ApoA1 to the protein or amino acid sequence in which they are present.

In one embodiment, there is provided the AAV vector according to the invention, wherein the HDL-binding moiety is an ApoA1-binding epitope derived from an ApoA1-binding protein selected from the group consisting of: PON1, LCAT, ABCA1, r587, q597, C1477 surround, S1506 surround and ApoB.

In one embodiment, there is provided the AAV vector according to the invention, wherein the ApoA1-binding epitope comprises an amino acid sequence with no more than 1 or 2 amino acid differences per 6 amino acids from an amino acid sequence selected from the group consisting of the amino acid sequences of SEQ ID NO's: 6-16, as presented in Table 1. Preferably, the ApoA1-binding epitope is expressed on all three of the VP1, VP2 and VP3 capsid proteins.

TABLE 1
ApoA1-binding epitopes integrated in the GH-L1 and/or the GH-L5 loops of
the AAV5 capsid proteins.

| Epitope origin | Amino acid sequence of HDL epitopes | SEQ ID NO |
|---|---|---|
| PON1 P1 | GKQLNLKL | SEQ ID NO: 6 |
| PON1 P2 | TLSEKAK | SEQ ID NO: 7 |
| LCAT s108 | TYSVEYLDSSK | SEQ ID NO: 8 |
| LCAT K249 | LKEEQR | SEQ ID NO: 9 |
| ABCA1 epitope 1 | RYVWGGFAYLQ | SEQ ID NO: 10 |
| epitope 1 r587 | FEDMRYVWG | SEQ ID NO: 11 |
| epitope 1 q597 | FAYLQDVVE | SEQ ID NO: 12 |
| ABCA1 epitope 2 | CPPGAGGLPPPQRKQNTADILQDLTGRNIS | SEQ ID NO: 13 |
| epitope 2 C1477 | MLPVCPPGAG | SEQ ID NO: 14 |
| surround | | |
| epitope 2 S1506 | GRNISDYLV | SEQ ID NO: 15 |
| surround | | |
| ApoB LDL receptor | RLTRKRGLK | SEQ ID NO: 16 |
| binding | | |

In one embodiment, there is provided the AAV vector according to the invention, wherein the ApoA1-binding epitope comprises an amino acid sequence with no more than 1 or 2 amino acid differences per 6 amino acids from an amino acid sequence selected from the group consisting of the amino acid sequences of SEQ ID NO's: 6-15. Preferably, the ApoA1-binding epitope is expressed on all three of the VP1, VP2 and VP3 capsid proteins.

In one embodiment, there is provided the AAV vector according to the invention, wherein the ApoA1-binding epitope comprises an amino acid sequence with no more than 1 or 2 amino acid differences per 6 amino acids from an amino acid sequence selected from the group consisting of the amino acid sequences of SEQ ID NO's: 6-14. Preferably, the ApoA1-binding epitope is expressed on all three of the VP1, VP2 and VP3 capsid proteins.

In one embodiment, there is provided the AAV vector according to the invention, wherein the ApoA1-binding epitope comprises an amino acid sequence with no more than 1 or 2 amino acid differences per 6 amino acids from an amino acid sequence selected from the group consisting of the amino acid sequences of SEQ ID NO's: 6, 8, 13 and 14. Preferably, the ApoA1-binding epitope is expressed on all three of the VP1, VP2 and VP3 capsid proteins.

In one embodiment, there is provided the AAV vector according to the invention, wherein the ApoA1-binding epitope comprises an amino acid sequence with no more than 1 or 2 amino acid differences per 6 amino acids from an amino acid sequence selected from the group consisting of the amino acid sequences of SEQ ID NO's: 8, 13 and 14. Preferably, the ApoA1-binding epitope is expressed on all three of the VP1, VP2 and VP3 capsid proteins.

In one embodiment, there is provided the AAV vector according to the invention, wherein the ApoA1-binding epitope comprises an amino acid sequence with no more than 1 or 2 amino acid differences per 6 amino acids from an amino acid sequence selected from the group consisting of the amino acid sequences of SEQ ID NO's: 6, 13 and 14. Preferably, the ApoA1-binding epitope is expressed on all three of the VP1, VP2 and VP3 capsid proteins.

In one embodiment, there is provided the AAV vector according to the invention, wherein the ApoA1-binding epitope comprises an amino acid sequence with no more than 1 or 2 amino acid differences per 6 amino acids from an amino acid sequence selected from the group consisting of the amino acid sequences of SEQ ID NO's: 8, 13 and 14. Preferably, the ApoA1-binding epitope is expressed on all three of the VP1, VP2 and VP3 capsid proteins.

In one embodiment, there is provided the AAV vector according to the invention, wherein the ApoA1-binding epitope comprises an amino acid sequence with no more than 1 or 2 amino acid differences per 6 amino acids from an amino acid sequence selected from the amino acid sequences of SEQ ID NO's: 13 and 14. Preferably, the ApoA1-binding epitope is expressed on all three of the VP1, VP2 and VP3 capsid proteins.

In one embodiment, there is provided the AAV vector according to the invention, wherein the ApoA1-binding epitope is inserted in at least one of the VP1, VP2 and VP3 capsid proteins, between positions corresponding to positions T444 and G445 and/or A579 and P580 of SEQ ID NO: 1, and wherein the ApoA1-binding epitope comprises an amino acid sequence with no more than 1 or 2 amino acid differences per 6 amino acids from an amino acid sequence selected from the group consisting of the amino acid sequences of SEQ ID NO's: 6-16. Preferably, the ApoA1-binding epitope is expressed on all three of the VP1, VP2 and VP3 capsid proteins.

In one embodiment, there is provided the AAV vector according to the invention, wherein the ApoA1-binding epitope is inserted in at least one of the VP1, VP2 and VP3 capsid proteins, between positions corresponding to positions T444 and G445 and/or A579 and P580 of SEQ ID NO: 1, and wherein the ApoA1-binding epitope comprises an amino acid sequence with no more than 1 or 2 amino acid differences per 6 amino acids from an amino acid sequence selected from the group consisting of the amino acid sequences of SEQ ID NO's: 6-15. Preferably, the ApoA1-binding epitope is expressed on all three of the VP1, VP2 and VP3 capsid proteins.

In one embodiment, there is provided the AAV vector according to the invention, wherein the ApoA1-binding epitope is inserted in at least one of the VP1, VP2 and VP3 capsid proteins, between positions corresponding to positions T444 and G445 and/or A579 and P580 of SEQ ID NO: 1, and wherein the ApoA1-binding epitope comprises an amino acid sequence with no more than 1 or 2 amino acid differences per 6 amino acids from an amino acid sequence selected from the group consisting of the amino acid sequences of SEQ ID NO's: 6-14. Preferably, the ApoA1-binding epitope is expressed on all three of the VP1, VP2 and VP3 capsid proteins.

In one embodiment, there is provided the AAV vector according to the invention, wherein the ApoA1-binding epitope is inserted in at least one of the VP1, VP2 and VP3 capsid proteins, between positions corresponding to positions T444 and G445 and/or A579 and P580 of SEQ ID NO: 1, and wherein the ApoA1-binding epitope comprises an amino acid sequence with no more than 1 or 2 amino acid differences per 6 amino acids from an amino acid sequence selected from the group consisting of the amino acid sequences of SEQ ID NO's: 6, 8, 13 and 14. Preferably, the ApoA1-binding epitope is expressed on all three of the VP1, VP2 and VP3 capsid proteins.

In one embodiment, there is provided the AAV vector according to the invention, wherein the ApoA1-binding epitope is inserted in at least one of the VP1, VP2 and VP3 capsid proteins, between positions corresponding to positions T444 and G445 and/or A579 and P580 of SEQ ID NO: 1, and wherein the ApoA1-binding epitope comprises an amino acid sequence with no more than 1 or 2 amino acid differences per 6 amino acids from an amino acid sequence selected from the group consisting of the amino acid sequences of SEQ ID NO's: 8, 13 and 14. Preferably, the ApoA1-binding epitope is expressed on all three of the VP1, VP2 and VP3 capsid proteins.

In one embodiment, there is provided the AAV vector according to the invention, wherein the ApoA1-binding epitope is inserted in at least one of the VP1, VP2 and VP3 capsid proteins, between positions corresponding to positions T444 and G445 and/or A579 and P580 of SEQ ID NO: 1, and wherein the ApoA1-binding epitope comprises an amino acid sequence with no more than 1 or 2 amino acid differences per 6 amino acids from an amino acid sequence selected from the group consisting of the amino acid sequences of SEQ ID NO's: 6, 13 and 14. Preferably, the ApoA1-binding epitope is expressed on all three of the VP1, VP2 and VP3 capsid proteins.

In one embodiment, there is provided the AAV vector according to the invention, wherein the ApoA1-binding epitope is inserted in at least one of the VP1, VP2 and VP3 capsid proteins, between positions corresponding to positions T444 and G445 and/or A579 and P580 of SEQ ID NO: 1, and wherein the ApoA1-binding epitope comprises an amino acid sequence with no more than 1 or 2 amino acid differences per 6 amino acids from an amino acid sequence selected from the amino acid sequences of SEQ ID NO's: 13 and 14. Preferably, the ApoA1-binding epitope is expressed on all three of the VP1, VP2 and VP3 capsid proteins.

In one embodiment, there is provided the AAV vector according to the invention, wherein the ApoA1-binding epitope is inserted in at least one of the VP1, VP2 and VP3 capsid proteins, between positions corresponding to positions T444 and G445 of SEQ ID NO: 1, and wherein the ApoA1-binding epitope comprises an amino acid sequence with no more than 1 or 2 amino acid differences per 6 amino acids from an amino acid sequence selected from the group consisting of the amino acid sequences of SEQ ID NO's: 6, 7, 8, 9, 13 and 14. Preferably, the ApoA1-binding epitope is expressed on all three of the VP1, VP2 and VP3 capsid proteins.

In one embodiment, there is provided the AAV vector according to the invention, wherein the ApoA1-binding epitope is inserted in at least one of the VP1, VP2 and VP3 capsid proteins, between positions corresponding to positions T444 and G445 of SEQ ID NO: 1, and wherein the ApoA1-binding epitope comprises an amino acid sequence with no more than 1 or 2 amino acid differences per 6 amino acids from an amino acid sequence selected from the group consisting of the amino acid sequences of SEQ ID NO's: 6, 7, 8, 13 and 14. Preferably, the ApoA1-binding epitope is expressed on all three of the VP1, VP2 and VP3 capsid proteins.

In one embodiment, there is provided the AAV vector according to the invention, wherein the ApoA1-binding epitope is inserted in at least one of the VP1, VP2 and VP3 capsid proteins, between positions corresponding to positions T444 and G445 of SEQ ID NO: 1, and wherein the ApoA1-binding epitope comprises an amino acid sequence with no more than 1 or 2 amino acid differences per 6 amino acids from an amino acid sequence selected from the group consisting of the amino acid sequences of SEQ ID NO's: 7, 8, 13 and 14. Preferably, the ApoA1-binding epitope is expressed on all three of the VP1, VP2 and VP3 capsid proteins.

In one embodiment, there is provided the AAV vector according to the invention, wherein the ApoA1-binding epitope is inserted in at least one of the VP1, VP2 and VP3 capsid proteins, between positions corresponding to positions T444 and G445 of SEQ ID NO: 1, and wherein the ApoA1-binding epitope comprises an amino acid sequence with no more than 1 or 2 amino acid differences per 6 amino acids from an amino acid sequence selected from the group consisting of the amino acid sequences of SEQ ID NO's: 6, 8, 13 and 14. Preferably, the ApoA1-binding epitope is expressed on all three of the VP1, VP2 and VP3 capsid proteins.

In one embodiment, there is provided the AAV vector according to the invention, wherein the ApoA1-binding epitope is inserted in at least one of the VP1, VP2 and VP3 capsid proteins, between positions corresponding to positions T444 and G445 of SEQ ID NO: 1, and wherein the ApoA1-binding epitope comprises an amino acid sequence with no more than 1 or 2 amino acid differences per 6 amino acids from an amino acid sequence selected from the group consisting of the amino acid sequences of SEQ ID NO's: 8 and 14. Preferably, the ApoA1-binding epitope is expressed on all three of the VP1, VP2 and VP3 capsid proteins.

In one embodiment, there is provided the AAV vector according to the invention, wherein the ApoA1-binding epitope is inserted in at least one of the VP1, VP2 and VP3 capsid proteins, between positions corresponding to positions T444 and G445 of SEQ ID NO: 1, and wherein the ApoA1-binding epitope comprises an amino acid sequence with no more than 1 or 2 amino acid differences per 6 amino acids from an amino acid sequence selected from the group consisting of the amino acid sequences of SEQ ID NO's: 13 and 14. Preferably, the ApoA1-binding epitope is expressed on all three of the VP1, VP2 and VP3 capsid proteins.

In one embodiment, there is provided the AAV vector according to the invention, wherein the ApoA1-binding epitope is inserted in at least one of the VP1, VP2 and VP3 capsid proteins, between positions corresponding to positions A579 and P580 of SEQ ID NO: 1, and wherein the ApoA1-binding epitope comprises an amino acid sequence with no more than 1 or 2 amino acid differences per 6 amino acids from an amino acid sequence selected from the group consisting of the amino acid sequences of SEQ ID NO's: 8, 13, 14 and 15. Preferably, the ApoA1-binding epitope is expressed on all three of the VP1, VP2 and VP3 capsid proteins.

In one embodiment, there is provided the AAV vector according to the invention, wherein the ApoA1-binding epitope is inserted in at least one of the VP1, VP2 and VP3 capsid proteins, between positions corresponding to positions A579 and P580 of SEQ ID NO: 1, and wherein the ApoA1-binding epitope comprises an amino acid sequence with no more than 1 or 2 amino acid differences per 6 amino acids from an amino acid sequence selected from the group consisting of the amino acid sequences of SEQ ID NO's: 8, 13 and 14. Preferably, the ApoA1-binding epitope is expressed on all three of the VP1, VP2 and VP3 capsid proteins.

In one embodiment, there is provided the AAV vector according to the invention, wherein the ApoA1-binding epitope is inserted in at least one of the VP1, VP2 and VP3 capsid proteins, between positions corresponding to positions A579 and P580 of SEQ ID NO: 1, and wherein the ApoA1-binding epitope comprises an amino acid sequence with no more than 1 or 2 amino acid differences per 6 amino acids from an amino acid sequence selected from the group consisting of the amino acid sequences of SEQ ID NO's: 13 and 14. Preferably, the ApoA1-binding epitope is expressed on all three of the VP1, VP2 and VP3 capsid proteins.

### Transgenes

In a further embodiment, there is provided an AAV vector according to the invention, wherein the AAV vector comprises a nucleic acid molecule encapsidated by the capsid proteins. In one embodiment, the nucleic acid molecule comprises a transgene flanked by at least one AAV inverted terminal repeat (ITR). Preferably, the ITRs are selected from a group consisting of AAV ITR sequences. More preferably, the ITRs sequences comprises the AAV1, AAV2, AAV5, AAV6, or AAV8 ITRs sequences. Optionally, when the nucleic acid molecule comprises two ITRs sequences, flanking the transgene on either side, the two ITRs are both AAV1, both AAV2, both AAV5, both AAV6, or both AAV8 ITRs sequences. Also optionally, said ITR sequence at the 5′ end of said DNA expression cassette differs from said ITR sequence at the 3′ of said DNA expression cassette, wherein said ITR sequence is one selected from the AAV1, AAV2, AAV5, AAV6 or AAV8 ITRs sequences.

Typically, the encapsidated nucleic acid molecule comprising the transgene, including ITRs and promoter, is 5,000 nucleotides (nt) or less in length. In another embodiment, an oversized DNA molecule, i.e. more than 5,000 nt in length, can be expressed in vitro or in vivo by using the AAV vector described by the present invention. An oversized DNA is here understood as a DNA exceeding the maximum AAV packaging limit of 5.5 kbp. Therefore, the generation of AAV vectors able to produce recombinant proteins that are usually encoded by larger genomes than 5.0 kb is also feasible.

The nucleotide sequence comprising the transgene as defined herein above may thus comprise a nucleotide sequence encoding a gene product of interest (for expression in the mammalian cell) or encoding a nucleotide sequence targeting a gene of interest (for silencing said gene of interest in a mammalian cell), and may be located such that it will be incorporated into an recombinant AAV vector replicated in a suitable production host cell. In the context of the invention it is understood that a particularly preferred mammalian cell in which the “gene product of interest” is to be expressed or silenced, is a human cell. Any nucleotide sequence can be incorporated for later expression in a mammalian cell transfected with the recombinant AAV vector produced in accordance with the present invention. The nucleotide sequence may e.g. encode a protein or it may express an RNAi agent, i.e. an RNA molecule that is capable of RNA interference such as, e.g. an shRNA (short hairpin RNA) or an siRNA (short interfering RNA). “siRNA” means a small interfering RNA that is a short-length double-stranded RNA that are not toxic in mammalian cells (Elbashir et al., 2001, Nature 411:494-98; Caplen et al., 2001, Proc. Natl. Acad. Sci. USA 98:9742-47). In a preferred embodiment, the nucleotide sequence comprising the transgene may comprise two coding nucleotide sequences, each encoding one gene product of interest for expression in a mammalian cell. Each of the two nucleotide sequences encoding a product of interest is located such that it will be incorporated into a recombinant parvoviral (rAAV) vector replicated in a suitable production host cell.

The product of interest for expression in a mammalian cell may be a therapeutic gene product. A therapeutic gene product can be a polypeptide, or an RNA molecule (si/sh/miRNA), or other gene product that, when expressed in a target cell, provides a desired therapeutic effect. A desired therapeutic effect can for example be the ablation of an undesired activity (e.g. VEGF), the complementation of a genetic defect, the silencing of genes that cause disease, the restoration of a deficiency in an enzymatic activity or any other disease-modifying effect. Examples of therapeutic polypeptide gene products include, but are not limited to growth factors, factors that form part of the coagulation cascade, enzymes, lipoproteins, cytokines, neurotrophic factors, hormones and therapeutic immunoglobulins and variants thereof. Examples of therapeutic RNA molecule products include miRNAs effective in silencing diseases, including but not limited to polyglutamine diseases, dyslipidemia or amyotrophic lateral sclerosis (ALS).

The diseases that can be treated using a recombinant parvoviral (rAAV) vector produced in accordance with the present invention are not particularly limited, other than generally having a genetic cause or basis. For example, the disease that may be treated with the disclosed vectors may include, but are not limited to, acute intermittent porphyria (AIP), age-related macular degeneration, Alzheimer's disease, arthritis, Batten disease, Canavan disease, Citrullinemia type 1, Crigler Najjar, congestive heart failure, cystic fibrosis, Duchene muscular dystrophy, dyslipidemia, glycogen storage disease type I (GSD-I), hemophilia A, hemophilia B, hereditary emphysema, homozygous familial hypercholesterolemia (HoFH), Huntington's disease (HD), Leber's congenital amaurosis, methylmalonic academia, ornithine transcarbamylase deficiency (OTC), Parkinson's disease, phenylketonuria (PKU), spinal muscular atrophy, paralysis, Wilson disease, epilepsy, Pompe disease, amyotrophic lateral sclerosis (ALS), Tay-Sachs disease, hyperoxaluria 9PH-1), spinocerebellar ataxia type 1 (SCA-1), SCA-3, u-dystrophin, Gaucher's types II or III, arrhythmogenic right ventricular cardiomyopathy (ARVC), Fabry disease, familial Mediterranean fever (FMF), proprionic acidemia, fragile X syndrome, Rett syndrome, Niemann-Pick disease and Krabbe disease. Examples of therapeutic gene products to be expressed include N-acetylglucosaminidase, alpha (NaGLU), Treg167, Treg289, EPO, IGF, IFN, GDNF, FOXP3, Factor VIII, Factor IX and insulin.

In one embodiment, there is provided an AAV vector according to the invention, wherein the AAV vector comprises a transgene encoding a human Factor IX. In a preferred embodiment, the transgene encodes the human Factor IX Padua variant, preferably a human Factor IX Padua variant comprising or consisting of the amino acid sequence of SEQ ID NO: 20, more preferably the transgene comprises the Factor IX coding sequence as comprised in SEQ ID NO: 21. In a further preferred embodiment, the AAV vector according to the invention comprises an expression cassette for the transgene encoding the human Factor IX or human Factor IX Padua variant, wherein i) the transgene is operably linked to a liver-specific promoter, preferably the P1 promoter, and/or ii) the transgene is operably linked to a polyadenylation site, preferably an SV40-derived polyA site. In yet a further preferred embodiment, the expression cassette is flanked by wt AAV2 ITRs. In one specific embodiment, an AAV vector according to the invention comprises or consists of the nucleotide sequence of SEQ ID NO: 21.

Alternatively, or in addition as another gene product, the nucleotide sequence comprising the transgene as defined herein above may further comprise a nucleotide sequence encoding a polypeptide that serves as a selection marker protein to assess cell transformation and expression. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel, supra. Furthermore, the nucleotide sequence comprising the transgene as defined herein above may comprise a further nucleotide sequence encoding a polypeptide that may serve as a fail-safe mechanism that allows to cure a subject from cells transduced with the recombinant parvoviral (rAAV) vector of the invention, if deemed necessary. Such a nucleotide sequence, often referred to as a suicide gene, encodes a protein that is capable of converting a prodrug into a toxic substance that is capable of killing the transgenic cells in which the protein is expressed. Suitable examples of such suicide genes include e.g. the E. coli cytosine deaminase gene or one of the thymidine kinase genes from Herpes Simplex Virus, Cytomegalovirus and Varicella-Zoster virus, in which case ganciclovir may be used as prodrug to kill the transgenic cells in the subject (see e.g. Clair et al., 1987, Antimicrob. Agents Chemother. 31: 844-849).

The nucleotide sequence comprising a transgene as defined herein above for expression in a mammalian cell, further preferably comprises at least one mammalian cell-compatible expression control sequence, e.g. a promoter, that is/are operably linked to the sequence coding for the gene product of interest, thus forming an expression cassette for expression of the gene product of interest in mammalian target cell to be treated by gene therapy with the gene product of interest. Many such promoters are known in the art (see Sambrook and Russel, 2001, supra). Constitutive promoters that are broadly expressed in many cell-types, such as the CMV promoter may be used. However, more preferred will be promoters that are inducible, tissue-specific, cell-type-specific, or cell cycle-specific. For example, for liver-specific expression (as disclosed in PCT/EP2019/081743) a promoter may be selected from an a1-anti-trypsin promoter, a thyroid hormone-binding globulin promoter, an albumin promoter, LPS (thyroxine-binding globin) promoter, HCR-ApoCII hybrid promoter, HCR-hAAT hybrid promoter and an apolipoprotein E promoter, LP1, HLP, minimal TTR promoter, FVIII promoter, hyperon enhancer, ealb-hAAT. Other examples include the E2F promoter for tumor-selective, and, in particular, neurological cell tumor-selective expression (Parr et al., 1997, Nat. Med. 3:1145-9) or the IL-2 promoter for use in mononuclear blood cells (Hagenbaugh et al., 1997, J Exp Med; 185:2101-10). In one embodiment, the promoter is a neurospecific promoter, such as a Neuron-Specific Enolase (NSE) promoter, a human synapsin 1 promoter, a cytomegalovirus/chicken beta-actin (CBA) promoter and a CaMKII kinase promoter.

The expression cassette for expression of the gene product of interest, as described above, further preferably encodes a polyA tail comprised in the DNA expression cassette operably linked to the 3′ end of the RNA molecule encoded by the transgene, as described above. Preferably, said polyA tail is the simian virus 40 polyadenylation (SV40 polyA), synthetic polyadenylation, Bovine Growth Hormone polyadenylation (BGH polyA).

Various modifications of the nucleotide sequences as defined above, including e.g. the wild-type AAV sequences, for proper expression in the host cell is achieved by application of well-known genetic engineering techniques such as described e.g. in Sambrook and Russell (2001) “Molecular Cloning: A Laboratory Manual (3rd edition), Cold Spring Harbor Laboratory, Cold Spring Harbor Laboratory Press, New York. Various further modifications of coding regions are known to the skilled artisan which could increase yield of the encode proteins. These modifications are within the scope of the present invention.

In one embodiment, any mammalian cell may be infected by an AAV vector of the invention, for example, but not limited to, a muscle cell, a liver cell, a nerve cell, a glial cell and an epithelial cell. In a preferred embodiment the cell to be infected is a human cell.

### Compositions

In another aspect, the invention pertains to a composition comprising an AAV vector according to the invention, i.e. an AAV vector comprising a capsid protein that comprises at least one HDL-binding moiety.

In one embodiment, the invention provides a composition comprising an AAV vector according to the invention and suitable excipients, such as buffers and stabilizers, antioxidant etc. In one particular embodiment these compositions are used to transduce cells in vitro or ex vivo, in which case the excipients will need to be compatible with cell culture.

In other preferred embodiments the compositions are used for treatment of (human) subjects. For that purpose the invention provides a pharmaceutical composition comprising an AAV vector according to the invention and at least one pharmaceutically acceptable carrier. In the case of AAV gene delivery vehicles a pharmaceutical composition typically comprise physiological buffers, such as e.g. PBS, comprising further stabilizing agents such as e.g. sucrose. Such compositions are compatible with and suitable and intended for use in subsequent intravenous, intrathecal, intraparenchymal, intravitreal, subretinal administration or for use in organ-targeted vascular delivery such as intraportal or intracoronary delivery or isolated limb perfusion.

### Uses

Another aspect of the invention relates to the use of an AAV vector according to the invention, or a composition comprising the AAV vector.

In one embodiment, there is provided an AAV vector according to the invention, or a composition comprising the AAV vector for use as a medicament.

In a further embodiment, there is provided an AAV vector according to the invention, or a composition comprising the AAV vector for use in the treatment of a condition that can be treated by gene therapy of the liver, kidneys, brain, pancreas, spleen, ovaries, testes or adrenals. More specifically, there is provided an AAV vector according to the invention, or a composition comprising the AAV vector for use in the treatment of a condition that can be treated by gene therapy of the liver. The liver is a central organ in metabolism. Consequently, numerous inherited metabolic disorders have their origin in this organ and can thus be treated by gene therapy of the liver. Such inborn errors of liver metabolism may lead to accumulation of toxic products in hepatocytes and extensive hepatotoxicity, as e.g. observed in disorders like a1-antitrypsin deficiency, type I tyrosinemia, or Wilson disease. In other metabolic diseases, such as in Crigler-Najjar syndrome type I, ornithine transcarbamylase deficiency, familial hypercholesterolemia, and hemophilia A and B, manifestations are primarily extrahepatic. The liver is therefore a target for gene therapy of such inborn errors of metabolism, of hemophilia A and B, and of acquired diseases such as liver cancer and hepatitis.

In a further embodiment, there is provided an AAV vector according to the invention, or a composition comprising the AAV vector for use in the treatment of hemophilia B, wherein preferably the AAV vector comprises a nucleic acid molecule comprising a transgene encoding a human Factor IX. In a preferred embodiment, the transgene encodes the human Factor IX Padua variant.

In a further embodiment, there is provided an AAV vector according to the invention, or a composition comprising the AAV vector for use in the treatment of a condition that can be treated by gene therapy of the central nervous system. Preferably, the condition is a condition that can be treated by gene therapy of the brain, more preferably the forebrain.

In a further embodiment, there is provided an AAV vector according to the invention, or a composition comprising the AAV vector for use in the treatment of a condition that can be treated by gene therapy of the cardiovascular system.

In a further embodiment, there is provided an AAV vector according to the invention, or a composition comprising the AAV vector for use in the treatment of a condition that can be treated by gene therapy of the gastrointestinal system.

In a further embodiment, there is provided an AAV vector according to the invention, or a composition comprising the AAV vector for use in the treatment of a condition that can be treated by gene therapy of the genitourinary system, such as the reproductive or urinary system.

### Nucleic Acid Molecules, Host Cells and Methods for Producing an AAV Vector

In one aspect, the invention pertains to a nucleic acid molecule comprising a nucleotide sequence encoding a capsid protein that comprises at least one HDL-binding moiety as defined herein above. In one embodiment, the nucleic acid molecule is an expression construct for expression of the capsid protein(s). Preferably, the expression construct is a construct for expression of the capsid protein(s) in a host cell that is suitable for the production of an AAV, such as a mammalian or insect cell line as further defined below.

Thus, in one embodiment, the expression construct for expression of the capsid protein(s) is an insect cell-compatible vector or a mammalian cell-compatible vector. An “mammalian cell-compatible vector” is understood to be a nucleic acid molecule capable of productive transformation or transfection of a mammalian cell or cell line. Mammalian cell-compatible vectors are well-known in the art. An “insect cell-compatible vector” is understood to be a nucleic acid molecule capable of productive transformation or transfection of an insect or insect cell. Exemplary insect cell-compatible vectors include plasmids, linear nucleic acid molecules, and recombinant viruses, such as baculoviruses. Any vector can be employed as long as it is insect cell-compatible. The mammalian or insect cell-compatible vector may integrate into the cell's genome but the presence of the vector in the cell need not be permanent and transient episomal vectors are also included. The vectors can be introduced by any means known, for example by chemical treatment of the cells, electroporation, or infection.

In one embodiment, the vector is a baculovirus, a viral vector, or a plasmid. In a more preferred embodiment, the insect cell-compatible vector is a baculovirus, i.e. the nucleic acid construct is a baculovirus-expression vector (BEV). It is well-known that baculovirus-expression vectors are particularly suitable for the transfer of nucleic acids to insect cells and methods for their use are described for example in: Summers and Smith, 1986, “A Manual of Methods for Baculovirus Vectors and Insect Culture Procedures”, Texas Agricultural Experimental Station Bull. No. 7555, College Station, Tex.; Luckow, 1991, In Prokop et al., “Cloning and Expression of Heterologous Genes in Insect Cells with Baculovirus Vectors' Recombinant DNA Technology and Applications”, 97-152; King and Possee, 1992, “The baculovirus expression system”, Chapman and Hall, United Kingdom; O'Reilly, Miller, and Luckow, 1992, “Baculovirus Expression Vectors: A Laboratory Manual”, New York; Freeman and Richardson, 1995, “Baculovirus Expression Protocols”, Methods in Molecular Biology, volume 39; U.S. Pat. No. 4,745,051; US2003148506; and WO 03/074714.

In one embodiment, there is provided at least one expression construct comprising separate expression cassettes for each of the VP1, VP2 and VP3 capsid proteins according to invention. In one embodiment, there is provided at least one expression construct comprising a separate expression cassette for a VP1 capsid proteins according to invention and a separate expression cassette for the VP2 and VP3 proteins according to invention. The various expression cassettes for the different capsid proteins according to invention can be present together on a single expression construct/nucleic acid molecule, or one or more of the expression cassettes for the different capsid proteins can be present on two or three separate expression constructs/nucleic acid molecules, each comprising one of the expression cassettes for the different capsid proteins. As will be understood, the use of separate expression cassettes for one or more of the different capsid proteins allows to produce AAV vectors with an HDL-binding moiety in only some of capsid proteins, e.g. only in VP1 and not in VP2 and VP3 or vice versa, or it allows to produce AAV vectors with different HDL-binding moieties in the different capsid proteins. Expression constructs for separate expression of the various capsid proteins in mammalian cells are e.g. disclosed in Judd et al. (Mol Ther Nucleic Acids. 2012; 1: e54). Expression constructs for separate expression of the various capsid proteins in insect cells are disclosed in co-pending application EP21177449.2.

In another embodiment, there is provided an expression construct comprising a single expression cassette for expression of all three of the VP1, VP2 and VP3 capsid proteins according to invention, preferably from a single coding sequence. Expression constructs for expression of all three of the VP1, VP2 and VP3 capsid proteins from a single expression cassette in mammalian cells are e.g. disclosed in Clark et al. (1995, Hum. Gene Ther. 6, 1329-134), Gao et al. (1998, Hum. Gene Ther. 9, 2353-2362), Inoue and Russell (1998, J. Virol. 72, 7024-7031), Grimm et al. (1998, Hum. Gene Ther. 9, 2745-2760) and Xiao et al. (1998, J. Virol. 72, 2224-2232). Expression constructs for expression of all three of the VP1, VP2 and VP3 capsid proteins from a single expression cassette in insect cells are e.g. disclosed in Urabe et al. (2002, Hum. Gene Ther. 13:1935-1943), WO2007/046703, WO2015/137802 and WO2019/016349. As will be understood, expression of all three of the VP1, VP2 and VP3 capsid proteins according to invention, from a single coding sequence (in a single expression cassette), allows to produce AAV vectors expressing an HDL-binding moiety in all three of its VP1, VP2 and VP3 capsid proteins.

In another aspect, the invention pertains to a host cell comprising a nucleic acid molecule or expression construct for expression of the capsid protein according to the invention. The host cell, preferably is a host cell that is suitable for the production of AAV vectors. Accordingly the host cell is a host cell that is amenable to in vitro culture, preferably at large scale. Host cell that are suitable for the production of AAV vectors are well-known in the art and will typically be a mammalian or an insect cell line. Mammalian cell lines for producing AAV vectors are selected from among any mammalian species, including, without limitation, cells such as A549, WEHI, 3T3, 10T1/2, BHK, MDCK, COS 1, COS 7, BSC 1, BSC 40, BMT 10, VERO, WI38, HeLa, a HEK 293 cell (which express functional adenoviral E1), Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumor cell. Mammalian cell lines for producing AAV vectors in particular include a broad range of HEK293 cell lines, of which the HEK293T cell line is preferred.

Insect cell lines for producing AAV vectors can be any cell line that is suitable for the production of heterologous proteins. Preferably the insect cell allows for replication of baculoviral vectors and can be maintained in culture, more preferably in suspended culture. In a preferred embodiment, the insect cell allows for replication of recombinant parvoviral vectors, including rAAV vectors. For example, the cell line used can be from Spodoptera frugiperda, Drosophila, or mosquito, e.g., Aedes albopictus derived cell lines. Preferred insect cells or cell lines are cells from the insect species which are susceptible to baculovirus infection, including e.g. S2 (CRL-1963, ATCC), Se301, SelZD2109, SeUCR1, Sf9, Sf900+, Sf21, BTI-TN-5B1-4, MG-1, Tn368, HzAm1, Ha2302, Hz2E5, High Five (Invitrogen, CA, USA) and expresSF+® (U.S. Pat. No. 6,103,526; Protein Sciences Corp., CT, USA).

In one embodiment, the host cell comprising the expression construct(s) for expression of the capsid protein(s) according to the invention, comprises further nucleotide sequences for the expression of an AAV vector. Such further nucleotide sequences typically include expression constructs for expression of AAV rep proteins in the host cell in question. Such further nucleotide sequences in addition usually include a nucleic acid construct comprising a transgene that is flanked by at least one AAV ITR sequence as described above.

In a further aspect, the invention relates to a method for producing an AAV vector of the invention, comprising capsid protein that comprises at least one HDL-binding moiety as defined herein above. The method preferably comprising the steps of: a) culturing a host cell as herein defined above under conditions such that the AAV vector is produced; and, b) optionally, one or more of recovery, purification and formulation of the AAV vector.

The AAV in the supernatant can be recovered and/or purified using suitable techniques which are known to those of skill in the art. For example, monolith columns (e.g., in ion exchange, affinity or IMAC mode), chromatography (e.g., capture chromatography, fixed method chromatography, and expanded bed chromatography), centrifugation, filtration and precipitation, can be used for purification and concentration. These methods may be used alone or in combination. In one embodiment, capture chromatography methods, including column-based or membrane-based systems, are utilized in combination with filtration and precipitation. Suitable precipitation methods, e.g., utilizing polyethylene glycol (PEG) 8000 and NH₃SO₄, can be readily selected by one of skill in the art. Thereafter, the precipitate can be treated with benzonase and purified using suitable techniques. In addition, recovery may preferably comprises the step of affinity-purification of the (virions comprising the) recombinant parvoviral (rAAV) vector using an anti-AAV antibody, preferably an immobilised antibody. The anti-AAV antibody preferably is a monoclonal antibody. A particularly suitable antibody is a single chain camelid antibody or a fragment thereof as e.g. obtainable from camels or llamas (see e.g. Muyldermans, 2001, Biotechnol. 74:277-302). The antibody for affinity-purification of rAAV preferably is an antibody that specifically binds an epitope on an AAV capsid protein, whereby preferably the epitope is an epitope that is present on capsid protein of more than one AAV serotype. E.g. the antibody may be raised or selected on the basis of specific binding to AAV2 capsid but at the same time also it may also specifically bind to AAV1, AAV3 and AAV5 capsids.

In general, suitable methods for producing an AAV vector according to the invention in mammalian or insect host cells, and means therefore (such as expression constructs for expression of AAV rep proteins), are described, for mammalian cells in: Clark et al. (1995, Hum. Gene Ther. 6, 1329-134), Gao et al. (1998, Hum. Gene Ther. 9, 2353-2362), Inoue and Russell (1998, J. Virol. 72, 7024-7031), Grimm et al. (1998, Hum. Gene Ther. 9, 2745-2760), Xiao et al. (1998, J. Virol. 72, 2224-2232) and Judd et al. (Mol Ther Nucleic Acids. 2012; 1: e54), and for insect cells in: Urabe et al. (2002, Hum. Gene Ther. 13:1935-1943), WO2007/046703, WO2007/148971, WO2009/014445, WO2009/104964, WO2011/122950, WO2013/036118, WO2015/137802, WO2019/016349 and in co-pending applications EP21177449.2, PCT/EP2021/058794 and PCT/EP2021/058798, all of which are incorporated herein in their entirety.

The present invention has been described above with reference to a number of exemplary embodiments as shown in the drawings. Modifications and alternative implementations of some parts or elements are possible, and are included in the scope of protection as defined in the appended claims.

### DESCRIPTION OF THE FIGURES

FIG. 1. SDS-PAGE gel ran with affinity batch binding materials obtained from purified AAV5-HDL batches that produced sufficiently concentrated material.

FIG. 2. Sequence of AAV5 VP1 annotated with the least conserved hypervariable regions among different AAV serotypes.

FIG. 3. The ability of the HDL capsids to in vitro transduce liver and neural cells lines was assessed in (A) Huh7, (B) HepG2, (C) Primary Human Hepatocytes (PHH) and (D) human iPSC-derived forebrain neuronal cells (i.e. forebrain-like neurons).

FIG. 4. In vitro secreted alkaline phosphatase (SEAP) reporter activity of AAV-HDL capsids of the invention in (a) Huh7 and (b) human IPSC-derived forebrain neuronal cells FIG. 5. ApoA1 sequence alignment and logo.

FIG. 6. Vector DNA copies in liver tissues obtained from C57BL/6-Tg(APOA1)1Rub/J mice injected with AAV-HDL capsids of the invention by QPCR.

FIG. 7. mRNA expression in various tissues obtained from C57BL/6-Tg(APOA1)1Rub/J mice injected with AAV-HDL capsids of the invention by RT-QPCR.

FIG. 8. SEAP reporter activity in citrated plasma obtained from C57BL/6-Tg(APOA1)1Rub/J mice injected with AAV-HDL capsids of the invention.

FIG. 9. Immunoprecipitation based assay used for characterizing the binding of AAV-HDL capsids of the invention to HDL in vitro.

### EXAMPLES

### 1. Introduction

In view of the increasing number of liver indications for which viable gene therapies are being developed, there is a need for improving AAV vector spread in the liver. HDL is one of the main lipid transporters that moves fatty acids, cholesterol and phospholipids from peripheral cells to the liver. As such the natural process can be used to carry AAV towards the liver. As explained elsewhere in this application, the same process may be used to carry AAV towards other HDL-associated organs, such as the kidneys, brain, pancreas, spleen, ovaries, testes and adrenals.

While HDL is a complex particle that consists of multiple proteins and phospholipids, its main protein component is ApoA1. ApoA1 (a biphasic protein) is a dimer that wraps around the phospholipids, presenting its apolar side to the lipids and its polar side to outside. ApoA1 also is the main docking molecule for proteins interacting with HDL during phospholipid loading. By introducing ApoA1-binding epitopes on the surface of an AAV capsid, including but not limited to an AAV5 capsid, we aim to increase the binding affinity of the AAV5 capsid to HDL, thereby exploiting the HDL particle as a carrier of the AAV towards the liver and/or other HDL-associated organs to improve its spread in said organs.

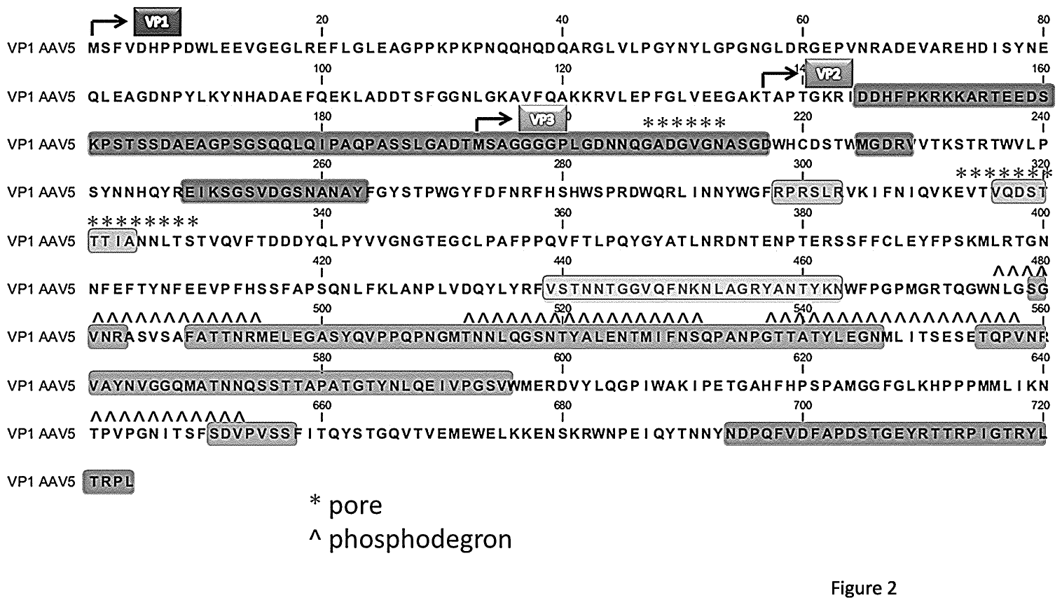

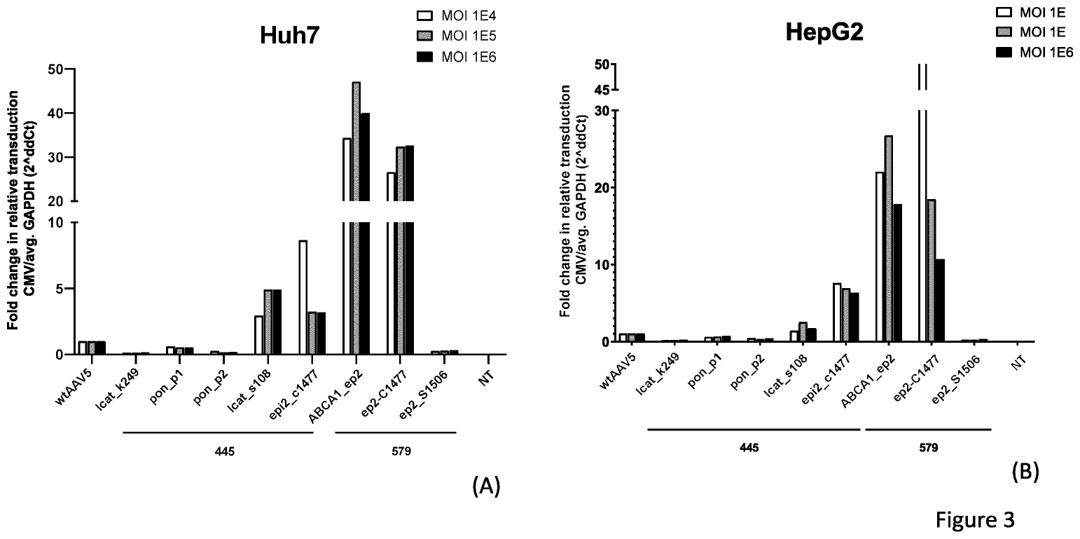

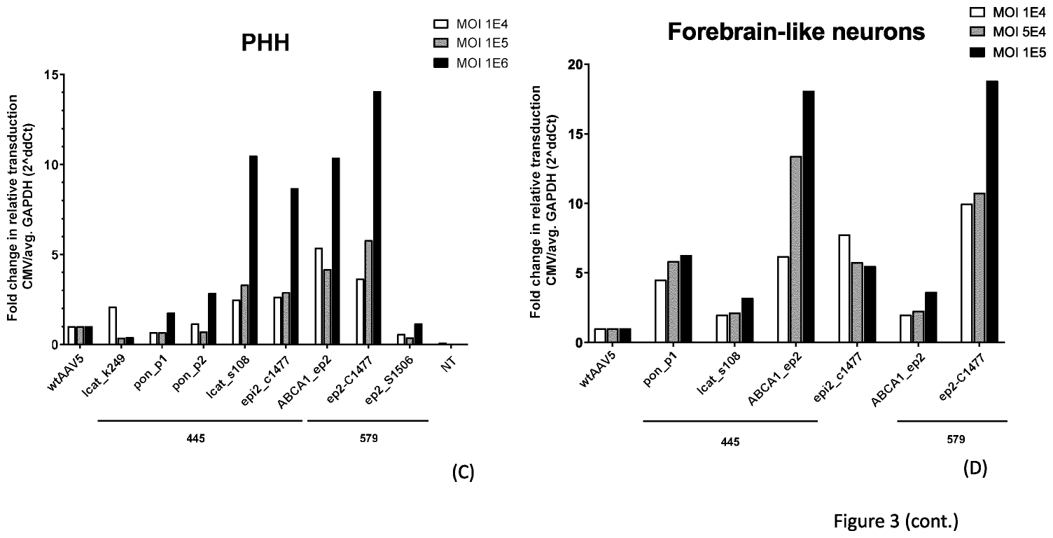

AAV is a virus that consists of 60 subunits (5×VP1, 5×VP2 and 50×VP3). When aligning multiple AAV serotypes, conserved and hypervariable regions can be identified across the capsid (FIG. 2). These hypervariable regions are known to be more accepting of insertions, as they are less likely to be involved in the structural foundation of the capsid. However, epitopes introduced on the capsid need to be surface exposed in order to be able to bind to HDL. Following mapping of the hypervariable regions to the 3D structure of the capsid (Govindasamy et al., J Virol. 2013, 87(20):11187-99. doi: 10.1128/JVI.00867-13; Zhang et al., Nat Commun. 2019, 10(1):3760. doi: 10.1038/s41467-019-11668-x), two surface exposed loops were selected for insertion of the HDL-binding epitopes: the GH-L1 loop and the GH-L5 loop. HDL-binding epitopes were inserted in the GH-L1 loop between positions 444 and 445 and in the GH-L5 loop between positions 579 and 580 of the wt AAV5 capsid sequence (SEQ ID NO. 1). Since both insertion sites are in VP3 section of the capsid cassette, and a single expression cassette is used for expression of all three VP1, VP2 and VP3 capsid proteins, each AAV particle will express 60 copies of an HDL-binding epitope exposed at its surface.

ApoA1 binding epitopes to be inserted into the AAV5 capsid protein were selected using the following criteria. First, ApoA1 binding proteins were identified from the Biogrid protein interaction database (95 in total, https://thebiogrid.org/106832/summary/homo-sapiens/apoa1.html). Second, ApoA1 binding proteins needed to have literature available that described the ApoA1-HDL binding interaction. Third, the proteins needed to have structural information available that described the exact amino acid sequence at which these interactions occurred. Using the criteria from above, three ApoA1-HDL interacting proteins were selected from which the HDL interaction epitopes were derived for insertion into the capsid coding sequence: 1) Pon1, an esterase associated with high density lipoproteins; 2) LCAT, an APOA1-activated enzyme that drives HDL maturation; and, 3) Abca1, a membrane protein associated with the transfer of cellular phospholipid and cholesterol to acceptor apolipoproteins, such as ApoA1. Additionally an ApoB binding epitope was included in the set. ApoB is a protein that is known to be associated with HDL and LDL and for which the receptor is located on liver cells. The ApoB LDL receptor binding epitope was recovered from https://www.uniprot.org/uniprot/P04114. The selected ApoA1-binding epitopes are summarized in Table 1.

For the avoidance of doubt, whilst the invention is exemplified using AAV5 capsid proteins, the delivery system of the invention may be used with capsid proteins from any other AAV serotypes. Without wishing to be bound by theory, and as explained above, incorporating HDL-binding proteins on the surface of AAV vectors of any serotype may be used to de-target the AAVs natural tropism, and re-target them towards HDL-associated organs, such as the liver and brain.

In a first example DNA sequences coding for the ApoA1-binding epitopes were integrated into the AAV5 capsid between amino acid positions 444 and 445, or between amino acid positions 579 and 580 of the wild-type (wt) AAV5 coding sequence. Next AAV5-HDL capsids were produced in Hek293t cells and purified with an affinity chromatography based protocol. After purification, AAV5-HDL capsids were subjected to quantitative (Virus titer by Q-PCR in gc/ml) and qualitative (Total protein gel) analysis. The ability of the HDL capsids to transduce liver and neural cells lines in vitro was assessed in Huh7, HepG2, human primary hepatocytes (PHH) and frontal neural brain cells (i.e. forebrain-like neurons or fore brain neurons). The ability of the AAV5-HDL capsids to drive Secreted Embryonic Alkaline Phosphatase (SEAP) reporter activity in vitro was also investigated.

In a second example, the performance of five AAV5-HDL capsids in C57BL/6-Tg(APOA1)1Rub/J mice was evaluated and compared to that of the wtAAV5 capsid. C57BL/6-Tg(APOA1)1Rub/J mice express human ApoA1. As the integrated epitopes originate from human proteins, it is unlikely that they will bind to ApoA1 originating from mice (FIG. 5). The following were evaluated: 1. vector genome copies in the liver that originate from the transgene packaged in the capsid; 2. SEAP reporter activity in mouse plasma driven by the AAV5-HDL and wtAAV5 capsids; and 3. transgene mRNA expression driven by AAV5-HDL capsids and wtAAV5 in a subset of tissues.

In a third example, the binding affinity of the AAV5-HDL capsids towards human HDL was investigated and compared against that of wtAAV5. The binding assay was performed in an in vitro setup and it was based on immunoprecipitation. AAV5-HDL or wtAAV5 were bound to HDL, the complex was precipitated and then the extend of the binding of the capsids to HDL was evaluated by QPCR.

### 2. Methods and Materials

### 2.1 Example 1: AAV5-HDL Production in Hek293t Cells and Affinity Purification

AAV5-HDL batches were produced with the AAV5 packaging system obtained from Vector Biolabs (VPK-405). Here, the plasmid containing the Cap gene (pRC5) was modified to include the HDL binding epitopes at aa445 (i.e., between positions T444 and G445 of SEQ ID NO: 1) or aa579 (i.e., between positions A579 and P580 of SEQ ID NO: 1). AAV5-HDL batches were produced in Hek293t cells (ATCC CRL-11268) by co-transfection of three plasmids using 1 mg/ml Polyethylenimine (Polysciences) transfection reagent. The transfected plasmids consisted of: 1) Cap AAV5-HDL/Rep plasmid; 2) Helper plasmid containing adenovirus helper sequences required for AAV production in Hek293t cells; and 3) Transgene plasmid containing a CMV-SEAP reporter flanked by wtAAV2 ITRs.

The AAV productions were performed at 37° C. in a 5% CO₂ humidified incubator. 72 hours after the transfection, cells were lysed for 1 hour by adding 10%, 10× lysis buffer (1.5 M NaCl, 0.5 M. Tris-HCl, 1 mM MgCl₂, 1% Triton x-100, pH 8.5) to the medium (DMEM+10% FBS). Next, genomic DNA was digested for 1 hour at 37° C. with 500 units benzonase (Merck)/25 ml culture. Cell lysate was clarified by centrifugation at 1900 g for 15 minutes, after which cell supernatant was stored at 4° C. until purification.

AAV5-HDL capsids were purified from cell lysates with an affinity chromatography protocol using AVB Sepharose (GE Healthcare). In brief, AVB Sepharose resin was washed in 0.2 M HPO₄ pH 7.5 buffer, after which cell lysate from the AAV production was added to the resin and incubated in a shaker incubator, shaking at 85 rpm for 2 hours at room temperature. Resin was washed again in 0.2 M HPO₄ pH 7.5 buffer. Next, bound virus was eluted from the resin with the addition of 0.2 M Glycine pH 2.5. pH-eluted virus was immediately neutralized with 0.5 M Tris-HCl PH 8.5.

### 2.2 Example 1: Virus Titration (Q-PCR) and SDS-Page Gel Electrophoresis of Purified AAV5-HDL Capsids

Viral titers of the purified AAV5-HDL material were determined by Q-PCR directed against the CMV promoter of the CMV-SEAP transgene. AAV-HDL capsids were subjected to Proteinase K treatment at 56° C. for 30 minutes after which they were diluted in a 16 ng/ml PolyA solution. Taqman Q-PCRs were with performed the following primer set: forward AATGGGCGGTAGGCGTGTA (SEQ ID NO: 17), reverse AGGCGATCTGACGGTTCACTAA (SEQ ID NO: 18) and probe Fam-TGGGAGGTCTATATAAGCAG-MGB (SEQ ID NO: 19). The QPCR was run on an Applied Biosystems Fast 7500 Real Time PCR system (Life Technologies). Results are presented in Table 2.

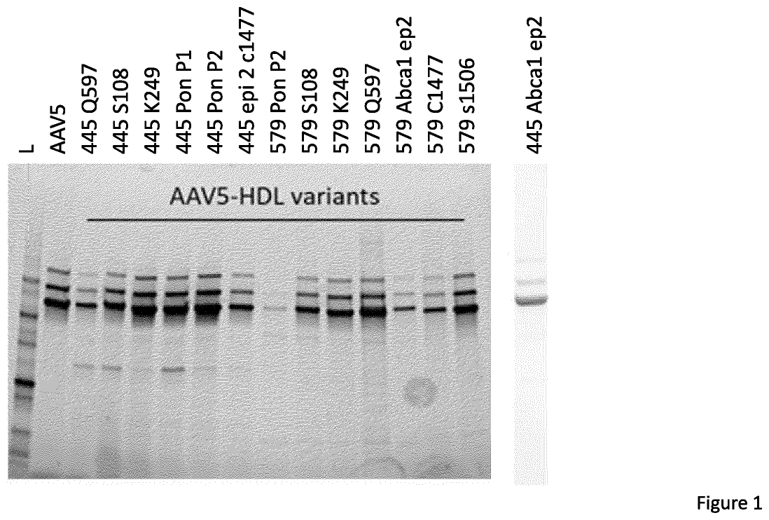

For SDS-PAGE gel electrophoresis of purified AAV5 capsids, 10 μl of batch binding purified AAV was loaded per lane of a 4-20% Mini-PROTEAN® TGX Stain-Free gel (Biorad). Following electrophoresis for 30 mins at 200 Volt in TGS buffer (Biorad), VP1, VP2 and VP3 bands were visualized under UV (280 nm) light on a Chemidoc Touch imaging system (Biorad). Results are presented in FIG. 1.

TABLE 2
Virus titers in genome copies per ml (gc/ml) obtained by qPCR from
AAV5-HDL crude cell lysates and batch binding purifications.

| Capsid | Lysate (gc/ml) | Purified (gc/ml) |
|---|---|---|
| AAV5 | 2.00E+12 | 3.20E+13 |
| 445 pon p1 | 4.00E+11 | 4.60E+12 |
| 445 pon p2 | 5.30E+11 | 8.30E+12 |
| 445 lcats108 | 3.70E+11 | 1.60E+12 |
| 445 lcat k249 | 7.30E+11 | 1.10E+13 |
| 445 Abca1 ep1 | 3.90E+09 | 1.63E+09 |
| 445 ep1 r587 | 3.10E+10 | 4.00E+10 |
| 445 ep1 q597 | 1.40E+10 | 7.20E+10 |
| 445 Abca1 ep2 | 1.94E+11 | 1.60E+12 |
| 445 ep2 c1477 | 4.80E+11 | 3.90E+12 |
| 445 Abca1 ep2 s1506 | 1.00e+11 | 5.10E+11 |
| 445 apob LDL | 8.80E+08 | 3.10E+09 |
| 579 pon1 p1 | 1.40E+10 | 3.20E+10 |
| 579 pon1 p2 | 2.00E+11 | 2.80E+12 |
| 579 lcat s108 | 2.00E+11 | 2.10E+12 |
| 579 lcat k249 | 2.30E+11 | 2.20E+12 |
| 579 Abca1 ep1 | 1.30E+09 | 9.70E+09 |
| 579 ep1 r587 | 7.80E+09 | 5.10E+10 |
| 579 ep1 q597 | 2.10E+11 | 2.20E+12 |
| 579 Abca1 ep2 | 1.40E+11 | 1.80E+12 |
| 579 ep2 C1477 | 1.50E+11 | 1.70E+12 |
| 579 ep2 S1506 | 2.20E+11 | 2.20E+12 |
| 579 apoB LDL | 1.20E+08 | 4.70E+08 |

### 2.3 Example 1: In Vitro Transduction of Huh7, HepG2, Human Primary Hepatocytes and Human iPSC-Derived Forebrain Neuronal Cells

The day before transduction, Huh7 and HepG2 were seeded in DMEM complemented with 10% FBS and without antibiotics, Primary human hepatocytes (PHH) were seeded on primary cell coated plated in hepatocytes plating medium without antibiotics. The cells were incubated with wt AAV5 or the different AAV5-HDL capsids at multiplicity of infection (MOI) close to 1E+03, 1E+04, 1E+05 or 1E+06. Forty-eight (48) hours post-transduction cell and culture supernatants were harvested. For Human iPSC-derived forebrain neurons, cells were seeded in Forebrain neuron maturation medium without antibiotics. The cells were incubated with wt AAV5 or different AAV5-HDL capsids at multiplicity of infection (MOI) close to 1E+03, 1E+04 or 1E+05. Sixty-eight (68) hours post-transduction cell and culture supernatants were harvested. Quantification of transduction was done using QPCR SybrGreen assay against the CMV promoter and GAPDH as loading control gene. The CMV expression relative to the control group (wt AAV5) was done by calculating the 2\hat{ }-ddCt. Results are presented in FIGS. 2A-D.

### 2.4 Example 1: In Vitro Secreted Alkaline Phosphatase Reporter Activity of AAV-HDL in Huh7 and Human IPSC-Derived Forebrain Neuronal Cells

The ability of AAV-HDL to drive SEAP reporter expression was evaluated in Huh7 and IPSC derived forebrain neuronal cells. 1e5 cells (both Huh7 and IPSC) were incubated for 48 hours with an MOI of 1e5 of AAV-HDL comprising a SEAP reporter gene. Transductions were performed in the presence of wild type adenovirus. Following the incubation, SEAP reporter activity was analyzed in the cell supernatant using the Phospha-Light™ SEAP Reporter Gene Assay System (Applied Biosystems). Luminescence, which is indicative of SEAP reporter activity, was measured on a Glowmax (Promega).

### 2.5 Example 2: In Vivo Performance of HDL Capsids in C57BL/6-Tg(APOA1)1Rub/J Mice

To examine the transduction efficacy and spread in multiple organs, C57BL/6-Tg(APOA1)1Rub/J mice were obtained from The Jackson Laboratory. Male C57BL/6-Tg(APOA1)1Rub/J mice were intravenously injected with the same dose (5e12 gc/kg) of AAV5 wt, AAV5-HDL 445_PON1 P1, AAV5-HDL 445_LCAT S108, AAV5-HDL 445_ABCA1 epitope 2, AAV5-HDL 445_epitope 2 C1477 and 579_epitope 2 C1477. An extra group of mice were injected with a four (4) fold higher concentration of AAV5 wt (2e13 gc/kg). The AAVs comprised a SEAP reporter gene. Another group of mice was treated with formulation buffer (PBS-0,014% (v/v) Tween2) and served as controls. Each group contained 8 animals. Blood samples were obtained after 4 hours fasting in week 0-day 1, week 2, 4 and week 6. The blood samples were treated with 10% v/v, 3.2% sodium citrate to obtain citrated plasma. Mice were sacrificed in week 6, after which multiple organs (liver, kidney, brain, pancreas, spleen, testes and adrenals) were collected for further analysis, as detailed below.

### 2.6 Example 2: Vector DNA Copies in Liver Tissues Obtained from C57BL/6-Tg(APOA1)1Rub/J Mice Injected with AAV-HDL by QPCR

Vector DNA copies in liver tissue (originating from the SEAP reporter gene) obtained from C57BL/6-Tg(APOA1)1Rub/J mice injected with AAV-HDL were determined by QPCR. In brief, liver tissue was pulverized on a Cryoprep crusher (Covaris) after which genomic DNA was isolated from the tissue using the DNeasy Blood and Tissue kit (Qiagen). Following elution of the genomic DNA from the kits spin columns, DNA concentrations were measured on a Nanodrop 2000 (ThermoFisher). To detect vector DNA copies in the sample, a QPCR specific for the CMV promoter was performed on 100 ng of genomic DNA. The QPCR was performed with primers: forward AATGGGGGGTAGGCGTGTA (SEQ ID NO: 17), reverse AGGCGATCTGACGGTTCACTAA (SEE ID NO: 18) and probe: FAM-TGGGAGGTCTATATAAGCAG-mgb (SEQ ID NO: 19) on a Quant Studio 5 Real time PCR system (ThermoFisher).

### 2.7 Example 2: SEAP Reporter Activity in Citrated Plasma Obtained from C57BL/6-Tg(APOA1)1Rub/J Mice Injected with AAV-HDL

The ability of AAV-HDL to drive SEAP reporter expression in vivo was evaluated in C57BL/6-Tg(APOA1)1Rub/J mice. Citrated plasma obtained from blood sampled on Week 0-day 1, week 2, week 4 and week 6 after injection was used to measure SEAP reporter activity. SEAP reporter activity was measured using the Phospha-Light™ SEAP Reporter Gene Assay System (Invitrogen™, T1017). Luminescence, which is indicative of SEAP reporter activity, was measured on a Glowmax (Promega).

### 2.8 Example 2: mRNA Expression in Various Tissues Obtained from C57BL/6-Tg(APOA1)1Rub/J Mice Injected with AAV-HDL by RT-QPCR

mRNA expression driven by the SEAP reporter was analysed in tissues obtained from C57BL/6-Tg(APOA1)1Rub/J mice injected with AAV5-HDL (with SEAP reporter). Liver, brain, heart, spleen and kidney tissues obtained from mice injected with wtAAV5, AAV5-HDL variants and vehicle were pulverized on a Cryoprep crusher (Covaris). Next, total RNA was isolated from the crushed tissue with the Direct-zol RNA miniprep kit (Zymo Research). RNA concentrations were quantified on a Nanodrop 2000 (ThermoFisher). RT reactions were setup using the Maxima first strand cDNA Synthesis kit (ThermoFisher) on 200 ng of Total RNA. This protocol included a DNAse treatment. mRNA copies were then determined by QPCR using primers specific for the SEAP gene (forward GTACCCAGATGACTACAGCCAAG (SEQ ID NO: 22) and reverse GGTGGATCTCGTATTTCATGTCT (SEQ ID NO: 23) on a Quant Studio 5 Real time PCR system (ThermoFisher).

### 2.9 Example 3: Assay Based on Immunoprecipitation Technique for Characterizing the Binding Between HDL and HDL Binding Capsids In Vitro

To evaluate whether the AAV5-HDL capsids bind to HDL in vitro, an immunoprecipitation-based assay was performed. 3e11 genome copies of either wtAAV5 or AAV5-HDL capsids were incubated with 25 ug of human HDL for 18 hours at room temperature while mixing at 25 rpm. The following day, HDL was precipitated with an antibody specific for ApoA1 (Abcam, ab52945) and the Classic Magnetic IP/Co-IP Kit (Pierce) using the protocol described in the kit. Complexed (AAV bound to HDL) and un-complexed (AAV not bound to HDL) AAV was then determined by QPCR. Q-PCRs were performed on a Fast 7500 Real Time PCR system (Applied Biosystems) using the following primer set: forward AATGGGCGGTAGGCGTGTA (SEQ ID NO: 24), rev AGGCGATCTGACGGTTCACTAA (SEQ ID NO: 25).

### 2.10 Example 3: Assay Based on Different Densities Between HDL and AAV for Characterizing the Binding Between HDL and HDL Binding Capsids

This assay is based on a difference in density between HDL (1.10 g/cm³) and AAV (1.21 g/cm³) particles. The idea behind this assay is that if AAV5-HDL and HDL particles interact, HDL will cover the surface of the capsid, thereby lowering the average density of the AAV particle. This density shift of the AAV particle can potentially be detected in a continuous iodixanol gradient, which can be created in an ultracentrifuge running at 342000×g for 20 hours. Here a HDL-HDL-AAV complex will be detected in a lighter fraction of the iodixanol gradient than an unbound wild type AAV. Iodixanol gradient fractions can be analyzed by QPCR for the presence of AAV. Plotting AAV titer of the fractions versus the density of that fraction can reveal a density shift of an AAV-HDL capsid vs unbound wt AAV5.

### 3. Results

### 3.1 Example 1: Quantitative and Qualitative Analysis of Purified AAV5-HDL Capsid

22 AAV5-HDL batches were produced cells by co-transfecting three plasmids required for AAV production (pRC5-AAV5-HDL, pHelper and pTransgene) into Hek293t. Following harvest of the cell lysates and affinity chromatography purification, the batches were subjected to quantitative analysis by qPCR to measure virus titer, and qualitative analysis by SDS-PAGE, to show integrity of the VP1, 2 and 3 capsid proteins. Table 2 summarizes the virus titers in gc/ml obtained in the crude cell lysates and in purified AAV5-HDL batches, respectively. Out of the 22 AAV5-HDL capsids that were designed (11 for amino acid insertion aa445 and 11 for amino acid insertion aa579), 14 were produced at sufficient concentration to perform SDS-PAGE analysis on. FIG. 1 displays the total protein gel obtained by running the purified AAV5-HDL batches produced with sufficient quantity on a SDS-PAGE gel. The total protein gel shows that the integrity of the AAV capsids with HDL binding epitope insertions remained intact, displaying clean VP1, 2 and 3 bands. Surprisingly we were able to insert an HDL binding epitope of 30 amino acids at both aa445 and aa579 (ABCA1 Epitope 2). An insertion of this length ranks among the longest insertions ever made in AAV5, when displayed in all three of VP1, VP2 and VP3, thereby being displaying 60 times on the capsid.

### 3.2 Example 1: In Vitro Transduction of Huh7, HepG2, Human Primary Hepatocytes and Human iPSC-Derived Forebrain Neuronal Cells

Huh7, HepG2, PHH and human iPSC-derived forebrain neurons were transduced with wt AAV5 or the different AAV5-HDL capsids for 48h for liver cell lines and for 72h for iPSC-derived forebrain neurons, respectively. FIG. 2 shows the fold change in relative transduction compared to wt AAV5 for the various AAV5-HDL capsid of respectively Huh7 cells (A), HepG2 cells (B), primary human hepatocytes (C) and iPSC-derived forebrain neurons (D). All of the ApoA1-binding epitopes tested are able to outperform wt AAV5 under at least some of the conditions tested. The AAV5-HDL 445_LCATs108, 445_epitope2 C1477, 579_ABCA1 epitope 2 and 579_epitope 2 C1477 capsids results in a better transduction up to 10- and 17-fold higher in primary human hepatocytes and iPSC-derived forebrain neurons, respectively, compared to AAV5 wt. In addition, AAV5-HDL 445_PON1 P1 and 445_ABCA1 epitope 2 (not tested in liver cell lines) shows better transduction specifically in human iPSC-derived forebrain neurons but not in the different liver cell lines.

### 3.3 Example 1: In Vitro SEAP Reporter Activity of AAV-HDL in Huh7 and Human IPSC-Derived Forebrain Neuronal Cells

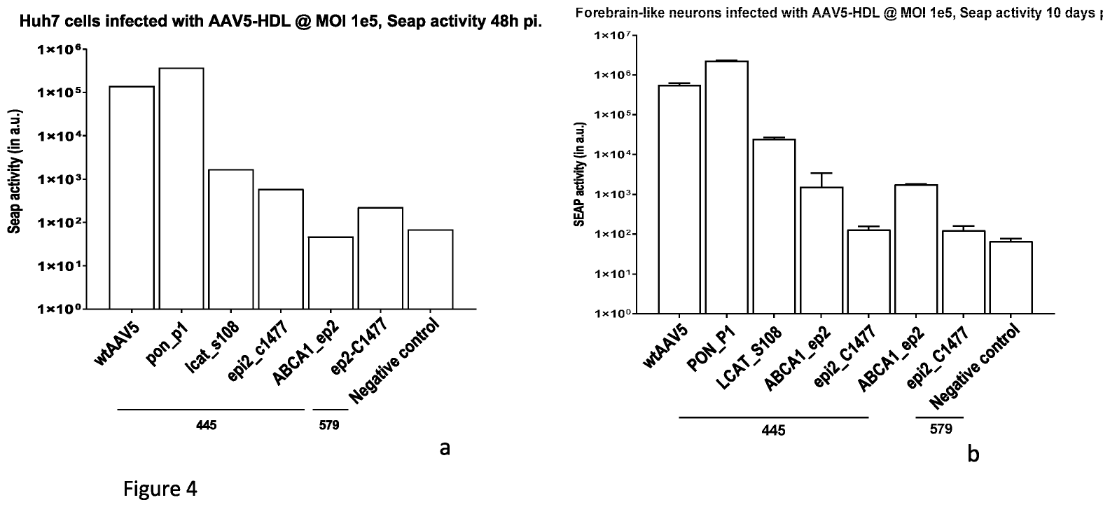

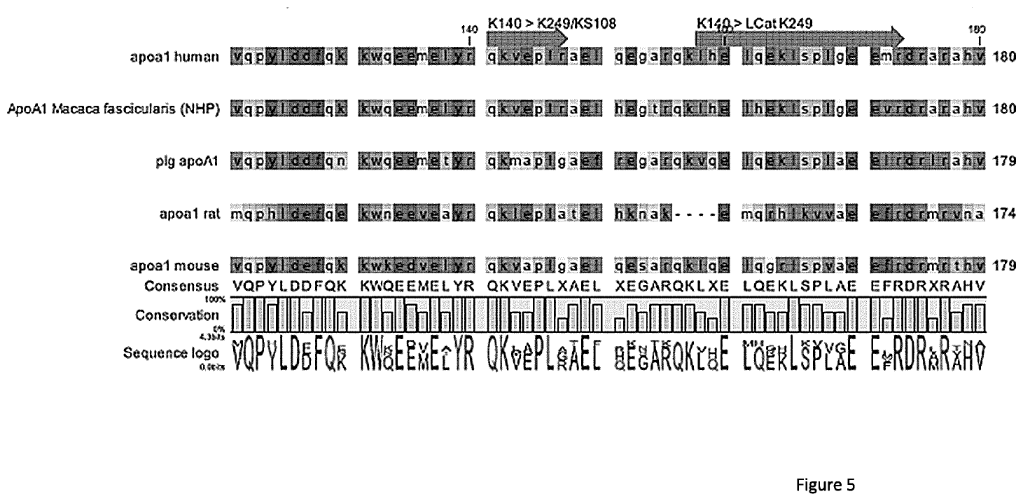

Huh7 cells and human IPSC-derived forebrain neurons were transduced with either wtAAV5 or AAV5 HDL capsids comprising a SEAP reporter gene. Both cell lines were infected at an MOI of 1e5 in the presence of wild type adenovirus for either 48 hours for the Huh7 cells or 10 days for the IPSC-derived forebrain neurons. Following the incubation, SEAP activity was measured in the cell supernatant (FIG. 4). Surprisingly, the AAV-HDL capsids which displayed high levels of transduction versus wtAAV5 in the in vitro transduction assay showed reduced SEAP reporter activity in both huh7 and IPSC-derived forebrain neurons. This reduced reporter activity was observed with HDL capsids AAV5-HDL 445_LCATs108, 445_epitope2 C1477, 579_ABCA1 epitope 2 and 579_epitope 2 C1477. Strikingly, and in contrast with the phenomenon observed with the other HDL capsids, HDL capsid 445 Pon P1, which in the transduction assay displayed similar to slightly improved transduction versus wtAAV5 (depending on the cell line), showed 2-3 fold improved reporter activity versus wtAAV5.

The discrepancy between the in vitro transduction and the SEAP reporter activity data indicates a potential impact of the origin of the HDL binding epitope integrated on the AAV5 capsid on the intracellular trafficking efficiency of the virus. This may in turn reflect on differences in the association strengths between HDL capsids and HDL.

### 3.4 Example 2: Vector DNA Copies in Liver Tissues Obtained from C57BL/6-Tg(APOA1)1Rub/J Mice Injected with AAV-HDL by QPCR

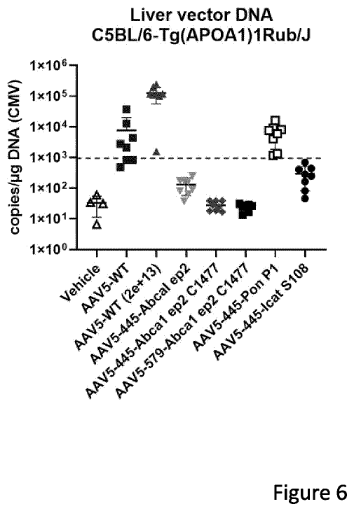

To investigate the in vivo performance of the HDL capsids, AAV5-HDL 445_LCATs108, 445_epitope2 C1477, 579_ABCA1 epitope 2, 579_epitope 2 C1477 and 445 Pon P1, as well as wtAAV5, were injected intravenously into C57BL/6-Tg(APOA1)1Rub/J mice at a dose of 5e12 gc/kg. Vector DNA copies per ug where determined in livers obtained at sacrifice, 6 weeks after the treatment. FIG. 6 shows the total count for each liver analysed. 445-Pon P1 (group 7) shows comparable results to the wtAAV5 (group 2) at the same injected concentration, whereas the total counts of HDL capsids AAV5-HDL 445_LCATs108, 445_epitope2 C1477, 579_ABCA1 epitope 2 and 579_epitope 2 C1477 where not detectable above levels found in the vehicle group (background).

### 3.5 Example 2: SEAP Reporter Activity in Citrated Plasma Obtained from C57BL/6-Tg(APOA1)1Rub/J Mice Injected with AAV-HDL

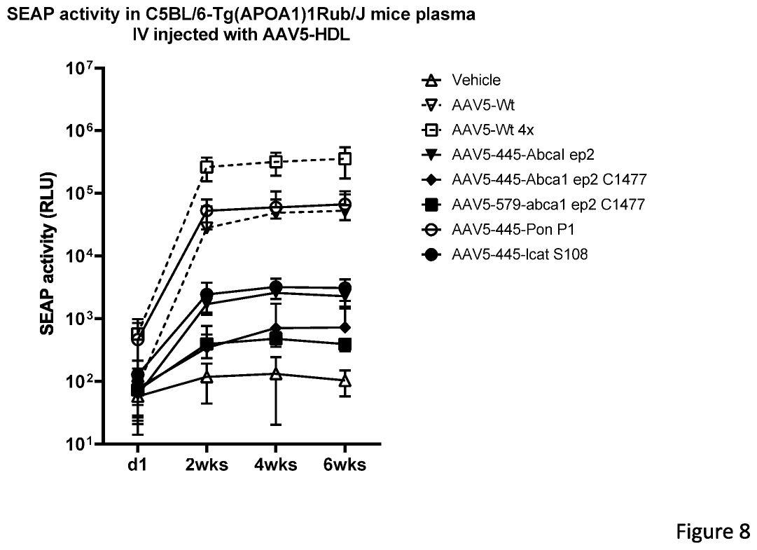

SEAP reporter activity was measured in citrated plasma samples obtained from C57BL/6-Tg(APOA1)1Rub/J mice injected intravenously with either HDL capsids or wtAAV5 comprising the SEAP reporter gene. Citrated plasma was obtained at the following timepoints after injection: Week 0-day 1, Week 2, Week 4 and Week 6. Mice injected with HDL capsids that did not display a measurable level of vector DNA copies in the liver (AAV5-HDL 445_LCATs108, 445_epitope2 C1477, 579_ABCA1 epitope 2 and 579_epitope 2 C1477) displayed various levels of low SEAP reporter activity compared to wtAAV5. Mice injected with HDL capsid 445 Pon P1 displayed similar to slightly improved levels of SEAP activity compared to wtAAV5 (˜50% improved versus wtAAV5 depending on the time point) (FIG. 8).

Interestingly, HDL capsid 445 Pon P1 displayed higher SEAP activity levels 1 day after injection compared to wtAAV5 administered at the same dose. This could potentially indicate that 445 Pon P1 and wtAAV5 infect different cell types in the liver, and/or a faster cellular transduction mechanism (e.g., cellular uptake, trafficking, uncoating, etc.). This observation could be substantiated by analysing the tissues for vector distribution by histological methods. Additionally the enhanced early expression of the SEAP reporter could also originate from tissues other than the liver. A broader analysis of mRNA expression driven by the SEAP reporter gene could enhance the understanding of vector distribution and expression driven by the HDL capsids.

### 3.6 Example 2: mRNA Expression in Various Tissues Obtained from C57BL/6-Tg(APOA1)1Rub/J Mice Injected with -AAV-HDL by RT-QPCR

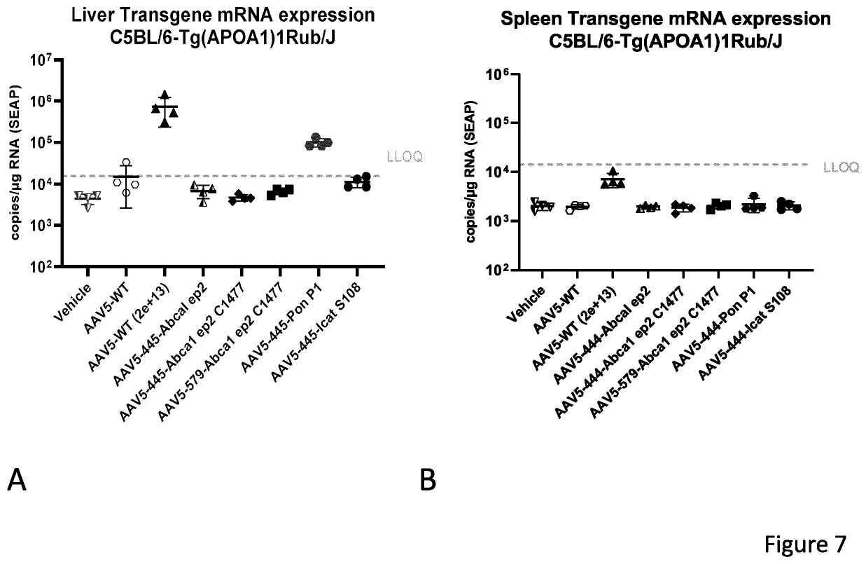

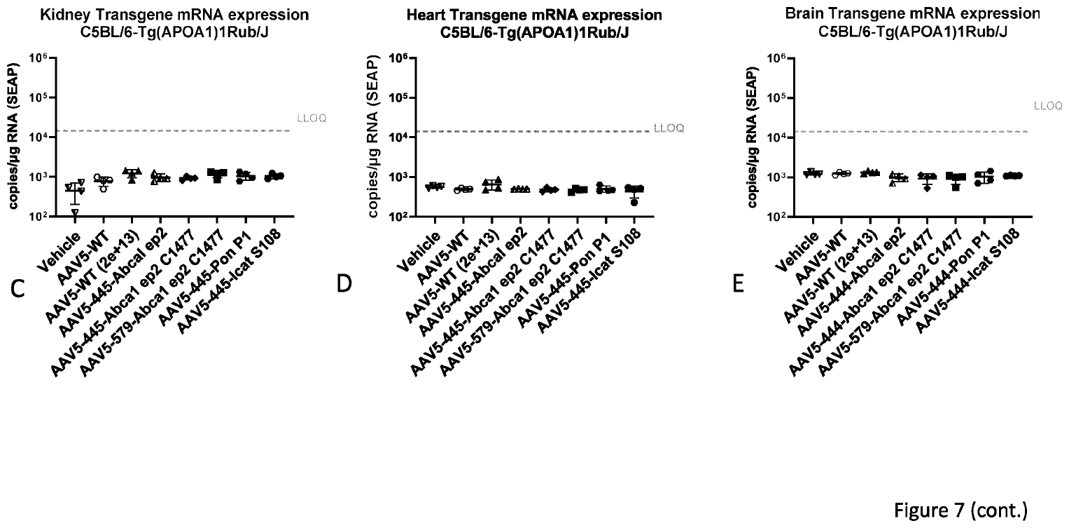

mRNA expression driven by the packaged SEAP reporter was investigated in various tissues obtained at sacrifice from C57BL/6-Tg(APOA1)1Rub/J mice injected intravenously with either HDL-AAV capsids or wtAAV5. FIG. 7 shows the copies of SEAP mRNA/ug in liver, spleen, kidney, heart and brain tissues, as measured with a QPCR specific for the SEAP gene. In the liver (FIG. 7A), low levels (close to the LLOQ) of SEAP mRNA expression were detected for HDL capsids AAV5-HDL 445_LCATs108, 445_epitope2 C1477, 579_ABCA1 epitope 2, 579_epitope 2 C1477. In contrast, SEAP mRNA copies driven by HDL capsid variant 445 Pon P1 fell between the mRNA levels detected in the wtAAV5 5e12 gc/kg and 2e13 gc/kg dose groups. mRNA levels for all HDL capsids were at levels close to the vehicle group in all other tissues (FIG. 7B-E). With the exception of the spleen, wtAAV5 (only at the 2e13 gc/kg dose) did not produce a measurable level of SEAP mRNA in tissues other than the liver. These results may be explained by the fact that a low dose of HDL AAV capsids was used. This dose was specifically selected within this experiment to highlight any expression differences between the capsids in the liver, where more substantial changes are expected.

Based on the vector DNA, SEAP reporter activity and the mRNA expression driven by the SEAP transgene, it can be concluded that HDL capsid variant 445 Pon P1 delivers higher transduction levels than wtAAV5 in the liver in vivo. This observation is supported by assays that show improved reporter expression at mRNA and protein activity level when compared to wtAAV5 injected at the same 5e12 gc/kg dose. Immunohistochemical analysis of liver tissues transduced with HDL capsid variant 445 Pon P1 may reveal if the origin of the stronger expression is the result of broader vector spread or a more efficient transduction of tissues.

### 3.7 Example 3: Assay Based on Immunoprecipitation Technique for Characterizing the Binding Between HDL and HDL Binding Capsids In Vitro

To evaluate whether binding occurs between the HDL capsids and human HDL, an in vitro binding assay based on the immunoprecipitation technique was performed. In the assay, HDL capsids (or wtAAV5) and human HDL were mixed and incubated for 18 hours at room temperature under gentle mixing. Following this incubation the (HDL)-(AAV-HDL) complex was precipitated from the solution by immunoprecipitation with an antibody specific for the HDL particle. If the HDL binding epitopes integrated on the surface of the AAV capsid facilitate improved binding of the capsid to HDL, there should be a higher amount of AAV in the precipitated HDL fraction of the HDL capsids compared to what is found in the wtAAV5 fraction. The different amounts of AAV in the bound (pulled down) and un-bound fractions (supernatant) can be measured with a QPCR.

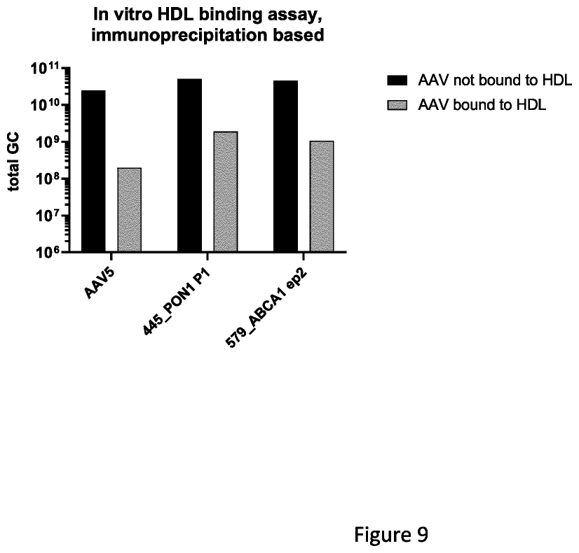

FIG. 9 shows results of the in vitro immunoprecipitation assay performed with wtAAV and HDL capsids 445-Pon P1 and 579-Abca1 ep2. The assay shows that, for both of the HDL capsids 445-Pon P1 and 579-Abca1 ep2, more AAV genome copies are found in the HDL bound fraction when compared to wtAAV5 (in both cases approximately 1 log more AAV genome copies). The highly similar levels of un bound AAV in all conditions (wtAAV and HDL capsids) indicates that equal amounts of AAV have been used as input in the experiment. The results of the in vitro experiment therefore indicate that there is an improved association of HDL with capsids that have a HDL binding epitope integrated on their surface (in this case, HDL capsids 445-Pon P1 and 579-Abca1 ep2).

1. An adeno-associated virus (AAV) vector comprising a capsid protein that comprises at least one high-density lipoprotein (HDL)-binding moiety, wherein preferably the HDL-binding moiety is exogenous to the capsid protein.

2. The AAV vector according to claim 1, wherein the capsid protein that comprises an HDL-binding moiety is at least a VP1 capsid protein, or wherein the capsid protein that comprise an HDL-binding moiety are at least the VP2 and VP3 capsid proteins.

3. The AAV vector according to claim 1, wherein the VP1, VP2 and VP3 capsid proteins each comprise an HDL-binding moiety.

4. The AAV vector according to claim 1, wherein at least one of:
a) an HDL-binding moiety is inserted into an exposed loop of the capsid protein, wherein preferably the exposed loop is at least one of the GH-L1 loop and the GH-L5 loop of the capsid protein; and,
b) an HDL-binding moiety is fused onto the carboxy-terminus of the capsid protein.

5. The AAV vector according to claim 1, wherein the HDL-binding moiety is an HDL-binding epitope or an HDL-binding protein or domain thereof, wherein preferably, the HDL-binding protein or domain thereof is an antibody, antibody fragment, scFv, Fv, Fab, (Fab′)2, single domain antibody (sdAb), vH or vL domain, camelid VHH domain, cartilaginous fish VNAR domain, DARPin, affibody, affilin, adnectin, affitin, repebody, fynomer, alphabody, avimer, atrimer, centyrin, pronectin or anticalin that specifically binds HDL.

6. The AAV vector according to claim 1, wherein the HDL-binding moiety has at least one of the characteristics:
a) the HDL-binding moiety, when inserted in the GH-L1 or the GH-L5 loop of the AAV5 VP1 amino acid sequence and expressed in at least the VP1 capsid protein of AAV5 capsids, after overnight incubation of the AAV5 capsids with an equimolar amount of human HDL, in an immunoprecipitation with an anti-human HDL antibody, causes the AAV5 capsids comprising the HDL-binding moiety to co-precipitate with human HDL for at least 10% of the AAV5 capsids or the HDL-binding moiety causes the ability of the AAV5 capsids comprising the HDL-binding moiety to co-precipitate with human HDL to increase by a factor 1.1, 1.2, 1.5, 2.0, 5.0, 10, 20, 50 or 100 as compared to corresponding wt AAV5 capsids;
b) when inserted in the GH-L1 or the GH-L5 loop of the AAV5 VP1 amino acid sequence and expressed in at least the VP1 capsid protein of AAV5 capsids, after overnight incubation of the AAV5 capsids with an equimolar amount of human HDL, in an continuous iodixanol density gradient centrifugation has a lighter density than the density of corresponding wt AAV5 capsids for at least 10% of the AAV5 capsids;
c) the HDL-binding moiety, when inserted in the GH-L1 or the GH-L5 loop of the AAV5 VP1 amino acid sequence and expressed in at least the VP1 capsid protein of an AAV5 vector, causes the AAV5 vector comprising the HDL-binding moiety to have an increased efficiency of transduction of at least a factor 1.1 as compared to a corresponding wt AAV5 vector when tested in vitro on a liver cell line or on primary human hepatocytes (PHH);
d) the HDL-binding moiety, when inserted in the GH-L1 or the GH-L5 loop of the AAV5 VP1 amino acid sequence and expressed in at least the VP1 capsid protein of an AAV5 vector, causes the AAV5 vector comprising the HDL-binding moiety to have an increased efficiency of transduction of at least a factor 1.1 as compared to a corresponding wt AAV5 vector when tested in vitro on human iPSC-derived forebrain neurons; and,
e) the HDL-binding moiety, when inserted in the GH-L1 or the GH-L5 loop of the AAV5 VP1 amino acid sequence and expressed in at least the VP1 capsid protein of an AAV5 vector, causes an increase in in vivo transduction of hepatocytes that are more distantly located from the portal vein by the AAV5 vector comprising the HDL-binding moiety as compared to a corresponding wt AAV5 vector, as determined in transgenic mice expressing human APOA1.

7. The AAV vector according to claim 1, wherein the HDL-binding moiety is an Apolipoprotein A-I (ApoA1)-binding moiety, wherein preferably the ApoA1-binding moiety is an ApoA1-binding epitope derived from an ApoA1-binding protein selected from the group consisting of: PON1, LCAT, ABCA1 and apoB.

8. The AAV vector according to claim 7, wherein the ApoA-1-binding epitope comprises an amino acid sequence with no more than 1 or 2 amino acid differences per 6 amino acids from an amino acid sequence selected from the group consisting of the amino acid sequences of SEQ ID NO's: 6-16, and wherein preferably, the ApoA1-binding epitope is inserted in at least one of the VP1, VP2 and VP3 capsid proteins, between positions corresponding to positions T444 and G445 of SEQ ID NO: 1 and/or between positions corresponding to positions A579 and P580 of SEQ ID NO: 1, wherein more preferably the ApoA-1-binding epitope is expressed on all three of the VP1, VP2 and VP3 capsid proteins.

9. The AAV vector according to claim 1, wherein at least one capsid protein is an AAV5 capsid protein.

10. The AAV vector according to claim 1, comprising a nucleic acid molecule encapsidated by the capsid proteins, wherein the nucleic acid molecule comprises a transgene flanked by at least one AAV inverted terminal repeat (ITR).

11. A composition comprising an AAV vector according to claim 1, wherein preferably the composition is a pharmaceutical composition comprising the AAV vector and at least one pharmaceutically acceptable carrier.

12.-15. (canceled)

16. A method for the treatment of a condition that can be treated by gene therapy of the liver or the central nervous system, the method comprising administering to a subject in need thereof an AAV vector according to claim 1.

17. A method for producing an AAV vector according to claim 1, the method comprising the steps of:
a) culturing a host cell comprising at least one HDL-binding moiety under conditions such that the AAV vector is produced; and,
b) optionally, one or more of recovery, purification and formulation of the AAV vector.
